(19) United States
(12) Patent Application Publication
(10) Pub. No.: US 2026/0007688 A1
(43) Pub. Date: Jan. 8, 2026

(54) SYNTHETIC ANALOGS OF CANNABINOL (CBN) FOR THE TREATMENT OF AGERELATED NEUROLOGICAL DISORDERS

(71) Applicant: Salk Institute for Biological Studies, La Jolla, CA (US)

(72) Inventors: Pamela A. Maher, La Jolla, CA (US); Zhibin Liang, San Diego, CA (US)

(73) Assignee: Salk Institute for Biological Studies, La Jolla, CA (US)

(21) Appl. No.: 19/259,937

(22) Filed: Jul. 3, 2025

Related U.S. Application Data
(63) Continuation of application No. PCT/US2024/010356, filed on Jan. 4, 2024.
(60) Provisional application No. 63/478,649, filed on Jan. 5, 2023.
(60) Provisional application No. 63/492,561, filed on Mar. 28, 2023.

(51) Int. Cl. A61K 31/00; A61P 25/28

(52) U.S. Cl. CPC A61K 31/658; A61P 25/28

(57) ABSTRACT

Disclosed herein are compounds having a structure according to Formula I

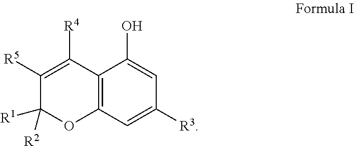

Also disclosed are methods for making and using the compounds. The compounds may be useful for treating or preventing a disease or condition in a subject, including a neurodegenerative disease or condition, a metabolic disorder, a traumatic brain injury, or cancer.

### CROSS REFERENCE TO RELATED APPLICATIONS

This application is a continuation of PCT/US2024/010356, filed Jan. 4, 2024, which claims the benefit of the earlier filing dates of U.S. provisional patent application No. 63/478,649, filed Jan. 5, 2023, and U.S. provisional patent application No. 63/492,561, filed Mar. 28, 2023, all of which are incorporated herein by reference in their entireties.

### FIELD

This application provides new cannabinol analogs, and methods of their use to treat age-related neurological disorders, metabolic disorders, traumatic brain injuries, and cancer.

### ACKNOWLEDGMENT OF GOVERNMENT SUPPORT

This invention was made with government support under AG064287 awarded by The National Institutes of Health. The government has certain rights in the invention.

### BACKGROUND

Cannabinol (CBN), a non-psychoactive cannabinoid from the cannabis plant, is a potent inhibitor of the oxytosis/ferroptosis pathway and can prevent mitochondrial dysfunction in neurodegeneration and cell aging independent of the cannabinoid CB1/CB2 receptor pathways.

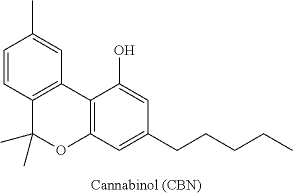 [SMILES: CCCCCc1cc(O)c2c(c1)OC(C)(C)c1ccc(C)cc1-2]

### SUMMARY

Disclosed herein are aspects of a method comprising administering a compound having a structure according to Formula I, or a pharmaceutically acceptable solvate or prodrug thereof, to a subject, for example, to treat or prevent a neurodegenerative disease or condition, a metabolic disorder, a traumatic brain injury, or cancer.

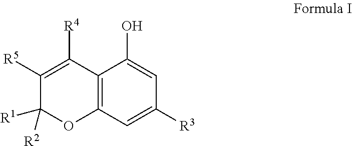

With respect to Formula I, each of R¹ and R² independently is hydrogen or alkyl, where at least one of R¹ and R² is not hydrogen; R³ is alkyl; and each of R⁴ and R⁵ is hydrogen or alkyl. In some aspects, both R⁴ and R⁵ are both hydrogen.

In some examples, each of R¹ and R² independently is hydrogen or C₁₋₆alkyl, such as hydrogen or methyl. In certain aspects, both of R¹ and R² are C₁₋₆alkyl, and both may be methyl.

In other aspects, R¹ is hydrogen and the compound has a structure according to Formula II or a pharmaceutically acceptable solvate or prodrug thereof.

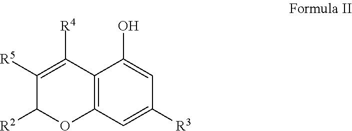

In such aspects, the compound may have a structure according to Formulas IIa or IIb or a pharmaceutically acceptable solvate or prodrug thereof.

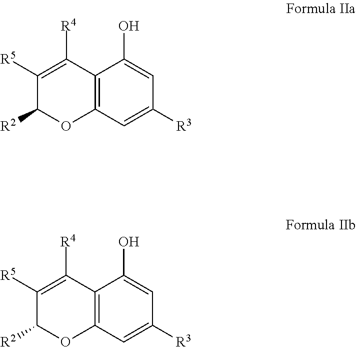

In some examples of Formulas IIa and IIb, R² is methyl.

In any aspects of Formulas I, II, IIa or IIb, R³ may be C₄₋₁₂alkyl, such as C₅-C₉alkyl and may be C₅-C₉ straight-chain alkyl, or C₅-C₉ branched alkyl. In particular examples, R³ is

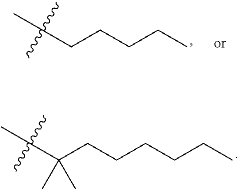

In some examples, the compound is administered as a pharmaceutical composition comprising the compound and a pharmaceutically acceptable carrier.

The foregoing and other objects and features of the disclosure will become more apparent from the following detailed description, which proceeds with reference to the accompanying figures.

### BRIEF DESCRIPTION OF THE DRAWINGS

FIG. 1 is a schematic diagram illustrating the fragment-based drug discovery approached used to determine the structure-activity relationship (SAR) of certain cannabinoid compounds.

FIG. 2 is a table providing the results from the oxytosis and ferroptosis assays for various compounds in the SAR analysis, with heatmap and symbol representation of percentage neuroprotection.

FIG. 3 is a table providing the results from the oxytosis and ferroptosis assays for various compounds in the SAR analysis, with heatmap and symbol representation of percentage neuroprotection.

FIG. 4 is a table providing the results from the oxytosis and ferroptosis assays for various compounds in the SAR analysis, with heatmap and symbol representation of percentage neuroprotection.

FIG. 5 is a table providing the results from the oxytosis and ferroptosis assays for various compounds in the SAR analysis, with heatmap and symbol representation of percentage neuroprotection.

FIG. 6 is a table illustrating the SAR results for various natural cannabinoids.

FIG. 7 provides structures of natural cannabinoids used in the SAR analysis having a free phenolic OH moiety.

FIG. 8 provides structures of natural cannabinoids used in the SAR analysis where the phenolic OH moiety is blocked.

FIG. 9 provides structures of natural cannabinoids used in the SAR analysis having a free phenolic COOH moiety.

FIG. 10 provides structures of additionally synthetic cannabinoids used in the SAR analysis.

FIG. 11 provides an exemplary pharmacophore derived from the SAR results against oxytosis and ferroptosis.

FIG. 12 is a graph of total ATP production rate versus compound, illustrating the total ATP production rate in HT22 cells after the different treatments for 16 hours.

FIG. 13 is a graph of compound versus percentage of the ATP production rate, illustrating the relative contribution of different compounds to ATP production from oxidative phosphorylation (OXPHOS) and glycolysis to total ATP production in HT22 cells.

FIG. 14 is a graph illustrating the levels of mitochondrial reactive oxygen species (ROS) in HT22 cells following treatment with the various compounds.

FIG. 15 is a schematic diagram illustrating the combinatorial chemistry approach to produce compounds CP1-CP4.

FIG. 16 provides representative micrographs of HT22 cells following different treatments for 16 hours: vehicle control, 50 nM RSL3, 50 nM RSL3+5 μM CBN, 50 nM RSL3+5 μM CP1, 50 nM RSL3+5 μM CP2, 50 nM RSL3+5 μM CP3, 50 nM RSL3+5 μM CP4. Micrographs show the representative morphological characteristics of the cell cultures under a given condition of 8 experimental replicates. Scale bar=200 μm.

FIG. 17 is a graph of C₁₁-BODIPY fluorescence of HT22 cells, illustrating the levels of cellular lipid peroxidation levels in HT22 cells following treatment with the various compounds for 16 hours.

FIG. 18 is a graph of mitochondrial oxygen consumption rate (OCR) versus time, illustrating the mitochondrial oxygen consumption rate profiles in HT22 cells in control and compound treated cells at 16 hours.

FIG. 19 is a graph of OCR versus respiration, illustrating basal respiration, maximal respiration, and spare respiration in control and compound treated HT22 cells.

FIG. 20 is the timeline for the Drosophila traumatic brain injury (mTBI) study.

FIG. 21 is a graph of percentage of dead flies versus treatment, illustrating the percentage of dead flies or mortality index (MI) for the different fly cohorts at 3 weeks following mTBI (10×) exposure.

### DETAILED DESCRIPTION

### I. Terms and Definitions

Unless otherwise noted, technical terms are used according to conventional usage. Definitions of many common terms in molecular biology may be found in Krebs et al. (eds.), Lewin's genes XII, published by Jones & Bartlett Learning, 2017; The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a compound” means “including a compound” without excluding other elements. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

In order to facilitate review of the various aspects of the disclosure, the following explanations of specific terms are provided:

About: Unless context indicated otherwise, “about” refers to plus or minus 5% of a reference value. For example, “about” 100 refers to 95 to 105. Unless otherwise indicated, all numbers expressing quantities of components, percentages, temperatures, times, and so forth, as used in the specification or claims, are to be understood as being modified by the term “about.”

Administration: To provide or give a subject an agent, such as a CBN analog described herein (such as anyone of compounds 1-4 disclosed herein), by any effective route. Administration can be local or systemic. Exemplary routes of administration include, but are not limited to, injection (such as subcutaneous, intramuscular, intradermal, intraperitoneal, intrathecal, intratumoral, intraosseous, intracerebral, and intravenous), oral, sublingual, rectal, transdermal, intranasal, vaginal and inhalation routes. In some examples, compounds provided herein are administered by intravenous injection.

Aliphatic: A substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and such arrangements including a cyclic component such as in —CH₂CH₂ (cyclopropyl), and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀), from one to six (C₁₋₆), or from one to four carbon atoms (C₁₋₄) for a saturated acyclic aliphatic group or moiety, from two to twenty-five carbon atoms (C₂₋₂₅); for example, from two to fifteen (C₂₋₁₅), from two to ten (C₂₋₁₀), from two to six (C₂₋₆), or from two to four carbon atoms (C₂₋₄) for an unsaturated acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety.

Alkyl: A saturated aliphatic hydrocarbyl group having from 1 to 25 (C₁₋₂₅) or more carbon atoms, more typically 1 to 10 (C₁₋₁₀) carbon atoms such as 1 to 6 (C₁₋₆) carbon atoms or 1 to 4 (C₁₋₄) carbon atoms. An alkyl moiety may be linear, branched, or cyclic, or may be linear or branched and include a cyclic portion, such as in-CH₂CH₂ (cyclopropyl). This term includes, by way of example, linear and branched hydrocarbyl groups such as methyl (—CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂ (CH₃)₂), sec-butyl (—CH(CH₃)(CH₂CH₃), t-butyl (—C(CH₃)₃), n-pentyl (—CH₂CH₂CH₂CH₂CH₃), neopentyl (—CH₂C(CH₃)₃), and 2-methyloctan-2-yl.

Alkenyl: An unsaturated aliphatic hydrocarbyl group containing one or more carbon-carbon double bonds (—C═C—) and having from 2 to 25 (C₂₋₂₅) or more carbon atoms, more typically 2 to 10 (C₂₋₁₀) carbon atoms such as 2 to 6 (C₂₋₆) carbon atoms or 2 to 4 (C₂₋₄) carbon atoms. An alkenyl moiety may be linear, branched, or cyclic, or may be linear or branched and include a cyclic portion.

Alkynyl: An unsaturated aliphatic hydrocarbyl group containing one or more carbon-carbon triple bonds and having from 2 to 25 (C₂₋₂₅) or more carbon atoms, more typically 2 to 10 (C₂₋₁₀) carbon atoms such as 2 to 6 (C₂₋₆) carbon atoms or 2 to 4 (C₂₋₄) carbon atoms. An alkynyl moiety may be linear, branched, or cyclic, or may be linear or branched and include a cyclic portion.

Cancer: A malignant solid or liquid tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Effective amount: The amount of an agent, such as one or more of the new CBN analogs disclosed herein (such as any of compounds 1˜4 disclosed herein), that is sufficient to effect beneficial or desired results. An effective amount (also referred to as a therapeutically effective amount) may vary depending upon one or more of: the subject and disease condition being treated, the weight and age of the subject, the severity of the disease condition, the manner of administration and the like. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one aspect, an “effective amount” of a therapeutic agent (e.g., one or more of the new CBN analogs disclosed herein, such as any of compounds 1-4 disclosed herein) is an amount sufficient to reduce the volume/size of a tumor, the weight of a tumor, the number of metastases, reduce the volume/size of a metastasis, the weight of a metastasis, or combinations thereof, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of one or more of the new CBN analogs disclosed herein, such as any of compounds 1˜4 disclosed herein). In one aspect, an “effective amount” of a therapeutic agent (e.g., one or more of the new CBN analogs disclosed herein) is an amount sufficient to reduce and/or ameliorate one or more signs or symptoms of an age-associated neurodegenerative disease (such as Alzheimer's disease or a related dementia) in a subject, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of one or more of the new CBN analogs disclosed herein, such as any of compounds 1˜4 disclosed herein). Signs or symptoms of an age-associated neurodegenerative disease that may be reduced and/or ameliorated by administration of one or more of the disclosed compounds may include, but are not limited to, decreases in memory, impairments in executive function, impairments in the ability to perform the activities of daily living, anxiety, depression, increases in blood or CSF markers of inflammation and increases in blood or CSF markers of neurodegeneration.

In some aspects an “effective amount” of a therapeutic agent (e.g., one or more of the new CBN analogs disclosed herein) is an amount sufficient to reduce signs or symptoms of a metabolic disorder (such as diabetes or a cardiovascular disease), for example by at least 5%, at least 10%, at least 15%, at least 20%, at least 25%, at least 30%, at least 40%, at least 50%, at least 60%, at least 70%, at least 75%, at least 80%, at least 90%, at least 95%, at least 98%, or even 100% (as compared to a suitable control, such as no administration of the therapeutic agent).

Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.

Pharmaceutically acceptable carrier: The pharmaceutically acceptable carriers useful in this disclosure are conventional. Remington's Pharmaceutical Sciences, 23rd Edition, Academic Press, Elsevier, (2020), describes compositions and formulations suitable for pharmaceutical delivery of a therapeutic agent, such as the CBN analogs disclosed herein.

In general, the nature of the carrier depends on the particular mode of administration being employed. For instance, parenteral formulations usually comprise injectable fluids that include pharmaceutically and physiologically acceptable fluids such as water, physiological saline, balanced salt solutions, aqueous dextrose, 5% human serum albumin, glycerol, or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Supplementary active compounds can also be incorporated into the compositions.

Example carriers include large, slowly metabolized macromolecules such as proteins, polysaccharides, polylactic acids, polyglycolic acids, polymeric amino acids, amino acid copolymers, liposomes, and inactive virus particles. Other exemplary carriers include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like.

Solvate: A complex formed by a combination of solvent molecules and a compound disclosed herein. The solvent can be an organic solvent, inorganic solvent, or a mixture thereof. The solvent may be water, in which case the solvate may be referred to as a hydrate. The disclosed compounds can exist as un-solvated compounds as well as in solvate forms, when combined with a solvent. Solvated forms of the presently disclosed compounds are within the scope of the aspects disclosed herein.

Prodrug: A chemical compound having undergone a chemical derivation such as substitution or addition of a further chemical group to change (for pharmaceutical use) any of its physico-chemical properties, such as solubility or bioavailability, e.g., ester and ether derivatives of an active compound that yield the active compound per se after administration to a subject. Examples of well-known methods of producing a prodrug of a given acting compound are known to those skilled in the art and can be found in, e.g., Krogsgaard-Larsen, et al. (2002) Textbook of Drug design and Discovery, Taylor & Francis.

Particularly favored prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species.

Subject: A vertebrate, such as a mammal, for example a human. Mammals include, but are not limited to, murines, simians, humans, farm animals, sport animals, and pets. In one aspect, the subject is a non-human mammalian subject, such as a monkey or other non-human primate, mouse, rat, rabbit, pig, goat, sheep, dog, cat, horse, or cow. In some examples, the subject has cancer (or a tumor), that can be treated using one or more of the new CBN analogs disclosed herein. In some examples, the subject has an age-associated neurodegenerative disease, such as Alzheimer's disease or a related dementia, that can be treated using one or more of the new CBN analogs disclosed herein. In some examples the subject has a metabolic disorder, such as diabetes (such as type II diabetes) or cardiovascular disease that can be treated using one or more of the new CBN analogs disclosed herein. In some examples, the subject is a laboratory animal/organism, such as a mouse, rabbit, or rat.

Tumor, neoplasia, or malignancy: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Certain compounds disclosed herein may exhibit tautomerism, conformational isomerism, geometric isomerism, and/or optical isomerism. For example, certain compounds disclosed herein may include one or more chiral centers and/or double bonds, and accordingly, may exist as stereoisomers, such as enantiomers or diastereomers, or a racemic mixture thereof, and/or geometric isomers (double-bond isomers). The disclosed compounds therefore may be provided as individual pure isomers, such as pure enantiomers or diastereomers, or as stereoisomeric mixtures, including racemic mixtures. In certain aspects, the compounds disclosed herein are synthesized in or are purified to be in substantially enantiopure form, such as in an 85% enantiomeric excess (e.e.), a 90% enantiomeric excess, a 95% enantiomeric excess, a 97% enantiomeric excess, a 98% enantiomeric excess, a 99% enantiomeric excess, or even in greater than a 99% enantiomeric excess, such as in a substantially enantiopure form. Additionally, with a compound comprising one or more asymmetric centers, one or both enantiomers or diastereomers are contemplated unless a specific enantiomer or diastereomer is shown or described.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by a ¹¹C- or ¹³C- or ¹⁴C-enriched carbon or ¹⁵N-enriched nitrogen, or ¹⁸F-enriched fluorine are within the scope of this invention.

### II. Overview

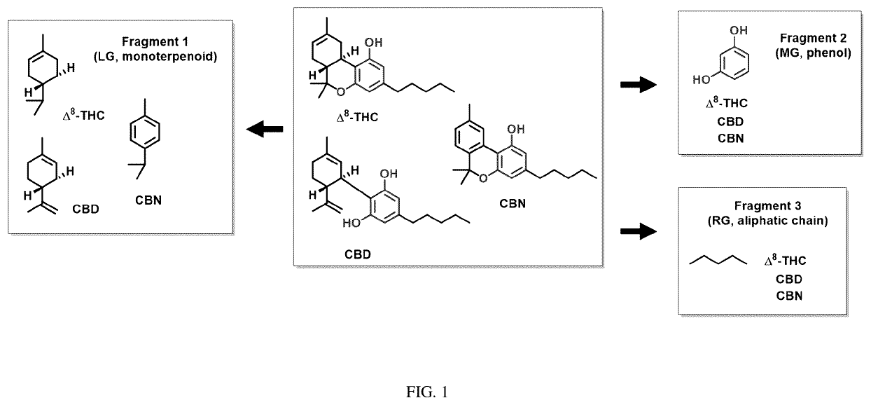

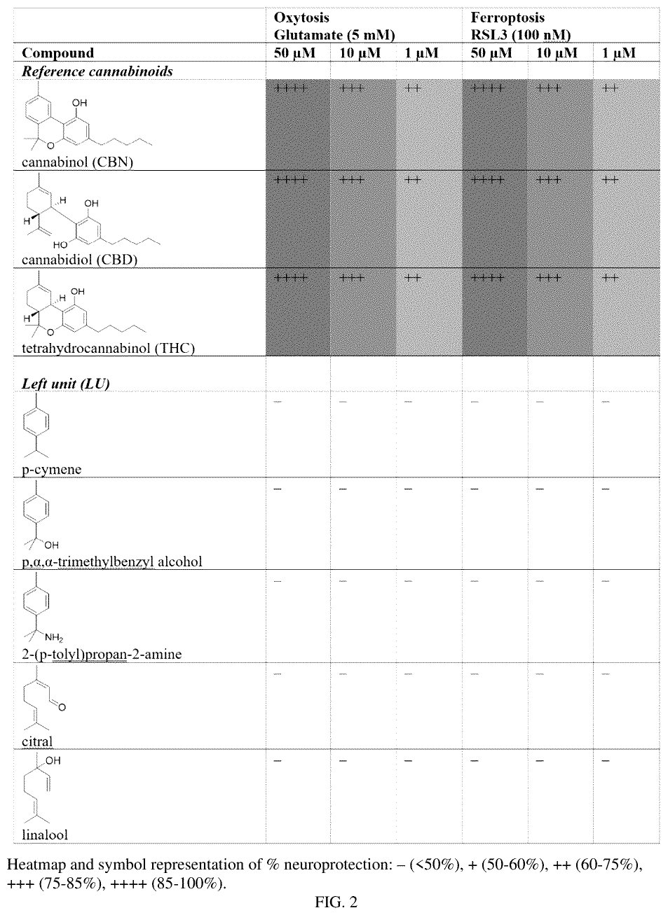

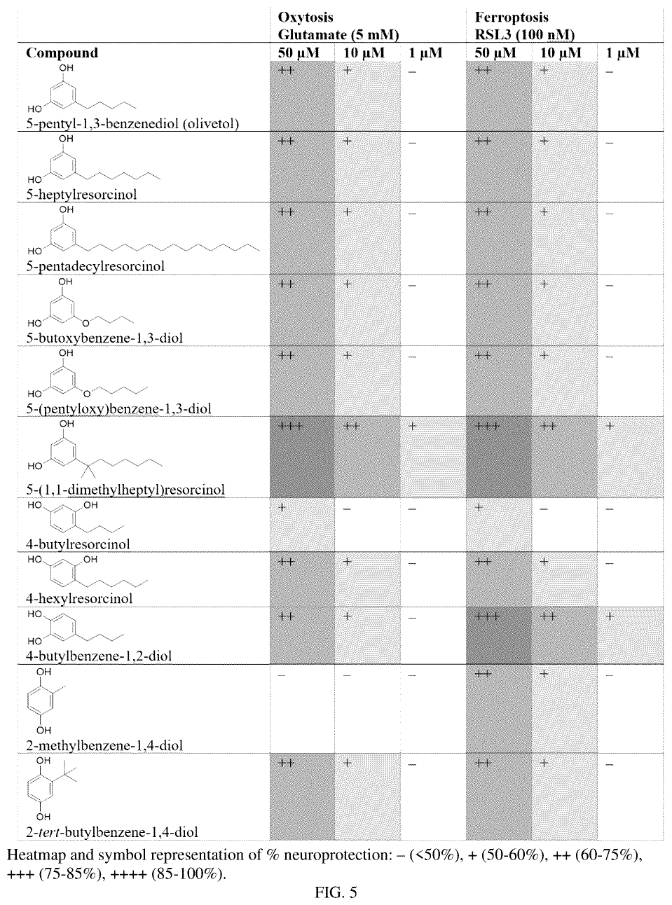

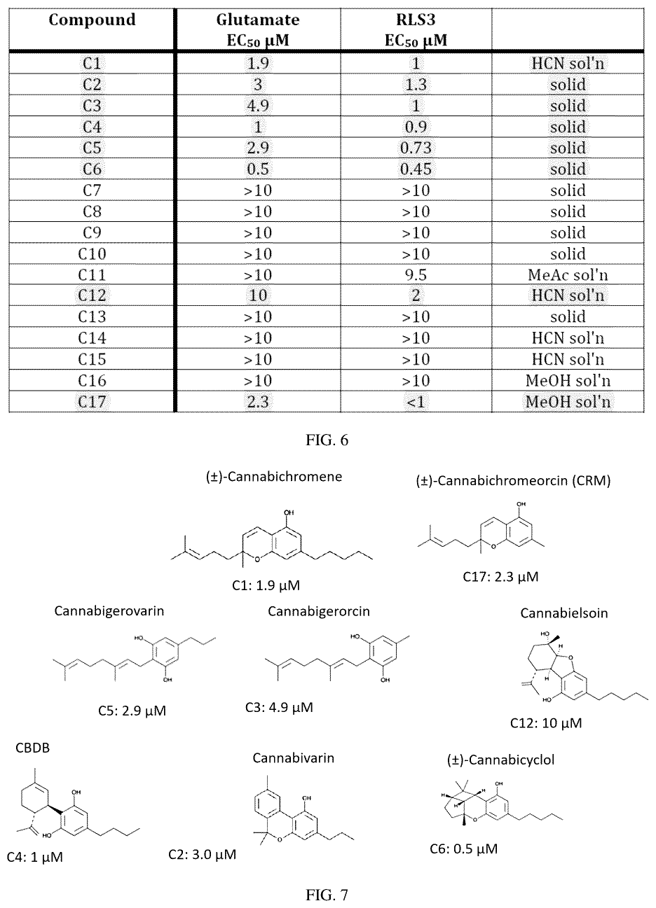

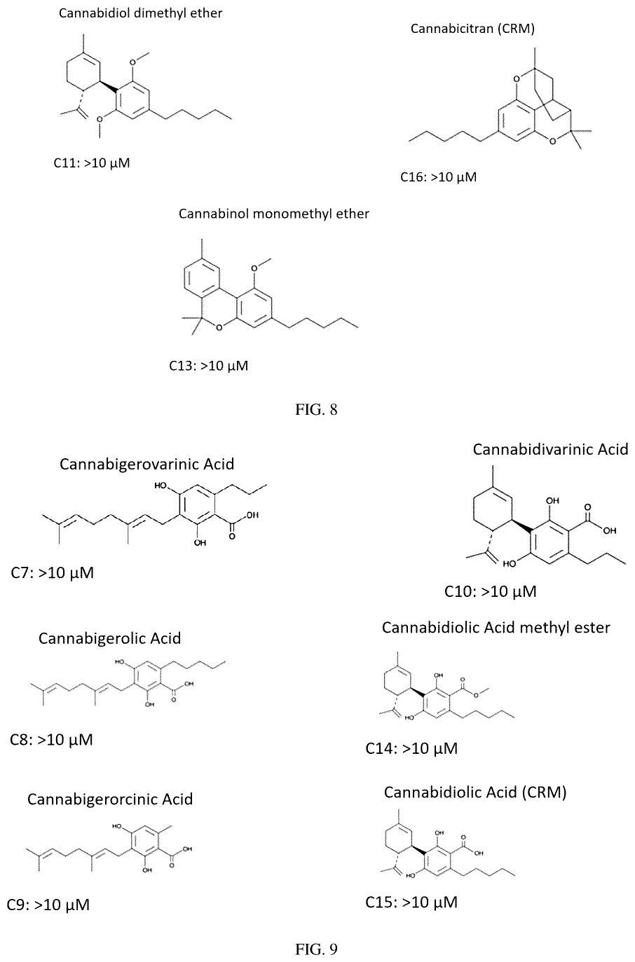

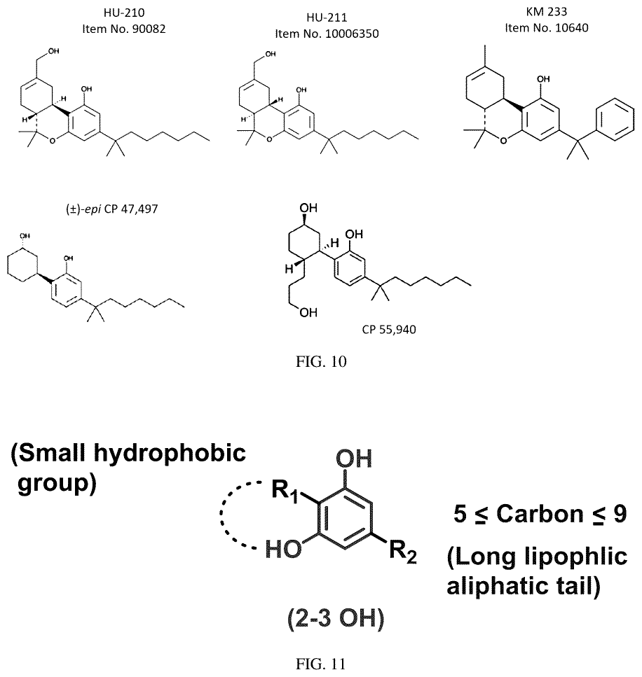

Using fragment-based drug discovery, preliminary structure-activity relationship (SAR) analyses demonstrated that functional antioxidant groups such as aromatic hydroxyls on the phytocannabinoids and their synthetic derivatives may be important for neuroprotection in cell-based assays. The structure of the cannabinoids was divided into three substructures (FIG. 1): a monoterpene fragment (left unit, fragment 1), a phenol fragment (central unit, fragment 2), and an aliphatic chain fragment (right unit, fragment 3). Each fragment was chemically simple and most of the substructures were commercially available. Therefore, a series of fragment chemicals with different variants of the substructures were screened in a cell-based phenotypic assays in the context of oxytosis/ferroptosis. Specifically, for fragment 1, monoterpenoids with different functional group and/or double-bond substitutions as well as ring arrangements were screened. For fragment 2, benzene rings with different functional group substitutions were screened. For fragment 3, different lengths or branches of the aliphatic hydrocarbon chains were examined. FIGS. 2-6 provide results from the various assays, with heatmap and symbol representation in FIGS. 2-5 indicating the percentage neuroprotection. And FIGS. 7-10 provide exemplary structures of compounds screened in the SAR analysis.

After the fragment chemicals were screened, the quantitative SAR, basic chemical building blocks, and pharmacophores were assessed to determine which, if any, are active alone. This information was then used to identify the most active and viable substructure for each fragment (FIG. 11). Combinations of substructures were used to form optimized CBN analogues through organic synthesis. The optimized CBN analogues were further validated in the same phenotypic assays used in the preliminary SAR studies for their potency and physiochemistry improvements, and then investigated for their in vivo efficacy in an animal model of aging and age-related neurological disease.

### III. Compounds

Disclosed herein are aspects of a cannabinol (CBN) analog compound having a structure according to Formula I

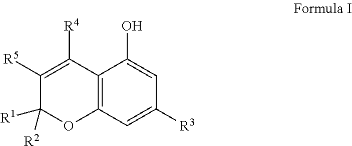

or a pharmaceutically acceptable solvate or prodrug thereof.

With respect to Formula I, each of R¹ and R² independently is hydrogen or alkyl, such as C₁-C₆alkyl, where at least one of R¹ and R² is not hydrogen. In some aspects, each of R¹ and R² independently is hydrogen or C₁-C₄alkyl, such as C₁-C₃alkyl. And in certain aspects, each of R¹ and R² independently is hydrogen, ethyl, or methyl, such as hydrogen or methyl.

In one example, one of R¹ and R² is hydrogen and the other of R¹ and R² is C₁-C₃alkyl, and may be methyl.

In another example, both R¹ and R² are C₁-C₃alkyl, and in certain aspects, both R¹ and R² are methyl.

R³ is aliphatic, such as alkyl, alkenyl or alkynyl, and may be C₄-C₁₂aliphatic, such as C₄-C₁₂alkyl, C₄-C₁₂alkenyl, or C₄-C₁₂alkynyl. In some aspects, R³ is C₄-C₁₂alkyl, such as C₄-C₁₀alkyl, C₅-C₁₀alkyl, or from C₅-C₉alkyl.

Each of R⁴ and R⁵ is hydrogen or alkyl, such as C₁-C₆alkyl, C₁-C₄alkyl, C₁-C₃alkyl, ethyl, or methyl. In some aspects, at least one of R⁴ and R⁵ is hydrogen, and in particular examples, both of R⁴ and R⁵ are hydrogen.

In some aspects, R¹ is hydrogen and the compound has a structure according to Formula II

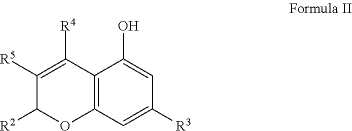

or a pharmaceutically acceptable solvate or prodrug thereof. With respect to Formula II, R³, R⁴ and R⁵ are as previously defined for Formula I and R² is alkyl, such as C₁-C₆alkyl, C₁-C₄alkyl, or C₁-C₃alkyl, and in certain examples, R² is methyl.

In some examples, of Formula II, the compound is a racemic mixture of optical isomers. In other examples, the compound has a structure according to Formula IIa or IIb, or a pharmaceutically acceptable solvate or prodrug thereof.

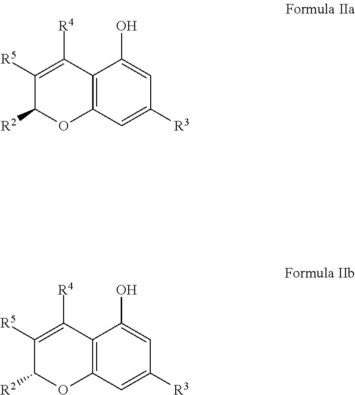

With respect to Formulas IIa and IIb, R², R³, R⁴ and R⁵ are as previously defined for Formula II.

In some aspects, the compound has a structure according to one of the following formulas, or a pharmaceutically acceptable solvate or prodrug thereof.

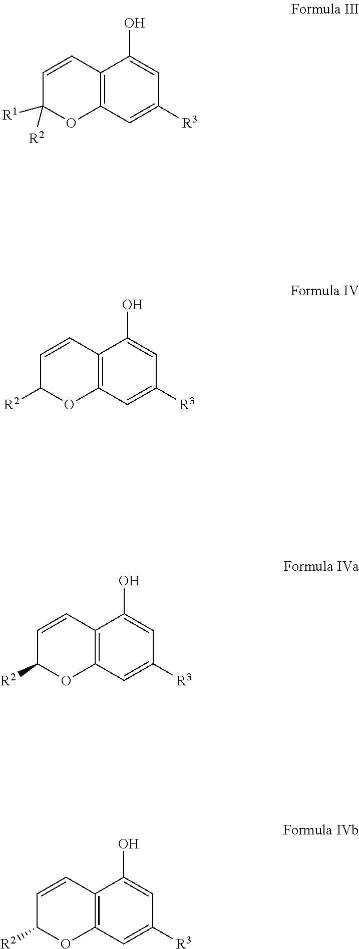

In some examples, R¹ is H and R² is methyl, or both of R¹ and R² are methyl. In such examples, the compound may have a structure according to one of the following formulas, or a pharmaceutically acceptable solvate or prodrug thereof.

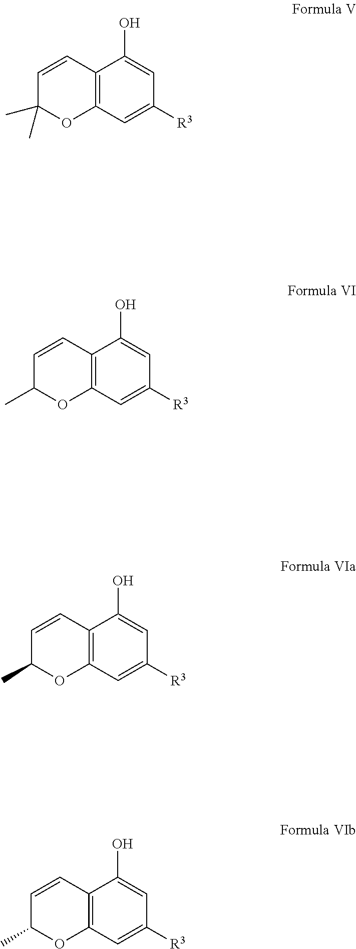

In any aspects of Formulas III to VIb, R¹, R², and R³, if present, are as defined previously for Formulas I and II.

In some aspects of Formulas I to VIb, R³ is C₅-C₉ straight-chain alkyl, such as C₅, C₆, C₇, C₈ or C₉ straight-chain alkyl. In other aspects of Formulas I to VIb, R³ is C₅-C₉ branched alkyl, such as C₅, C₆, C₇, C₈ or C₉ branched alkyl. In certain examples, R³ is

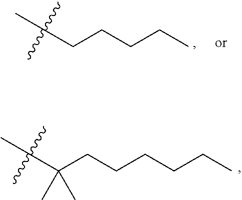

where ‘ [symbol] ’ indicates the point of attachment to the rest of the molecule.

Some exemplary compounds according to formula I include:

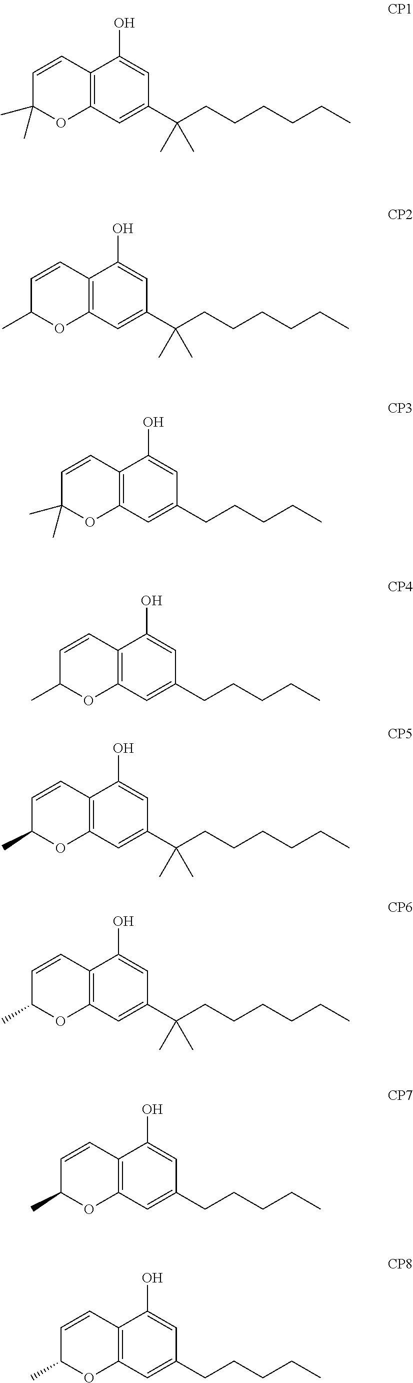

Exemplary compounds according to formula I include:
CP1: 2,2-dimethyl-7-(2-methyloctan-2-yl)-2H-chromen-5-ol;
CP2: 2-methyl-7-(2-methyloctan-2-yl)-2H-chromen-5-ol;
CP3: 2,2-dimethyl-7-pentyl-2H-chromen-5-ol;
CP4: 2-methyl-7-pentyl-2H-chromen-5-ol;
CP5: (S)-2-methyl-7-(2-methyloctan-2-yl)-2H-chromen-5-ol;
CP6: (R)-2-methyl-7-(2-methyloctan-2-yl)-2H-chromen-5-ol;
CP7: (S)-2-methyl-7-pentyl-2H-chromen-5-ol; or
CP8: (R)-2-methyl-7-pentyl-2H-chromen-5-ol.

### IV. Synthesis

Disclosed compounds can be prepared as exemplified below, as illustrated for specific compounds in the examples, and as will be understood by persons of ordinary skill in the art of organic synthesis. An exemplary synthesis may comprise the reaction step shown in Scheme 1.

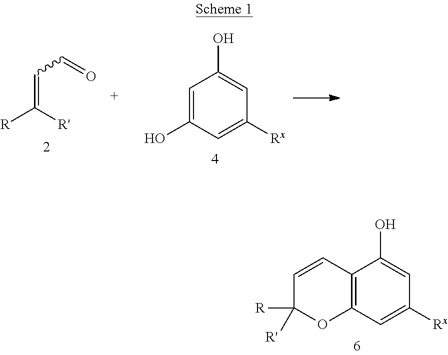

With respect to Scheme 1, R and R′ independently is H or alkyl, where typically at least one of R and R′ is alkyl. And R^{x} is alkyl. Compound 4 is treated with compound 2 in the presence of a base in a suitable solvent to form compound 6. The base can be any base suitable to facilitate the reaction. In some aspects, the base is an alkyl amine base, such as dimethylamine, trimethylamine, diethylamine, triethylamine, n-propylamine, n-butylamine, or a combination thereof. Typically, the solvent is an aprotic solvent, such as, but not limited to, toluene, acetonitrile, DMF, or combinations thereof. The reaction mixture is heated to a suitable temperature for a period to time to facilitate the reaction proceeding towards completion. The temperature may be from 50° C. to 150° C. or more, and maybe at the reflux temperature of the solvent. After the reaction has cooled, the product is isolated and may be purified, such as by chromatography.

### V. Methods of Use

### A. Diseases and Conditions

The compounds disclosed herein (such as any one of compounds 1˜4 disclosed herein) can be used to treat or prevent diseases or conditions in a subject, such as a neurodegenerative disease or condition. In some aspects, the subject exhibits one or more risk factors or symptoms associated with a neurodegenerative disease or condition or the development of a neurodegenerative disease or condition. In some aspects, the neurodegenerative disease or condition is an age-associated neurodegenerative disease or condition.

Examples of neurodegenerative diseases or conditions that may be treated or prevented by administration of one or more of the disclosed compounds (such as any one of compounds 1˜4 disclosed herein) include, but are not limited to, Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), traumatic brain injury (TBI), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, or Icelandic hereditary cerebral hemorrhage with amyloidosis. In some aspects, the neurodegenerative disease or condition is Parkinson's disease, Alzheimer's disease, prion disease, a motor neuron disease (MND), amyotrophic lateral sclerosis (ALS), or Huntington's disease (HD).

Additionally, or alternatively, the disclosed compounds may be useful for treating or preventing a disease or disorder associated with mitochondrial dysfunction associated with an aging brain in a subject. In some aspects, the subject exhibits one or more risk factors or symptoms associated with a disease or disorder associated with mitochondrial dysfunction associated with an aging brain or the development of a disease or disorder associated with mitochondrial dysfunction associated with an aging brain.

In some aspects, the disease or disorder associated with mitochondrial dysfunction associated with an aging brain is Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), traumatic brain injury (TBI), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, or Icelandic hereditary cerebral hemorrhage with amyloidosis. In some aspects, the disease or disorder associated with mitochondrial dysfunction associated with an aging brain is selected from Parkinson's disease, Alzheimer's disease, prion disease, a motor neuron disease (MND), amyotrophic lateral sclerosis (ALS), or Huntington's disease (HD).

Also disclosed herein are aspects of a method for administering a compound disclosed herein (such as any one of compounds 1˜4 disclosed herein) to a subject, where administration of the compound results in one or more of suppression of mitochondrial oxidative stress in oxytosis/ferroptosis (such as a reduction of at least 20%, at least 50%, at least 75%, at least 90%, at least 95%, at least 99%, or 100%, for example as compared to such mitochondrial oxidative stress in oxytosis/ferroptosis prior to administration of the compound(s) or as compared to no administration of the compound(s)), maintenance of mitochondrial calcium homeostasis in oxytosis/ferroptosis, modulation of oxidative phosphorylation system, restoration of mitochondrial bioenergetics (such as an increase of at least 20%, at least 50%, at least 75%, at least 90%, at least 100%, at least 200%, or at least 500%, for example as compared to such mitochondrial bioenergetics prior to administration of the compound(s) or as compared to no administration of the compound(s)), promotion of mitochondrial biogenesis (such as an increase of at least 20%, at least 50%, at least 75%, at least 90%, at least 100%, at least 200%, or at least 500%, for example as compared to such mitochondrial bioenergetics prior to administration of the compound(s) or as compared to no administration of the compound(s)), reduction of intraneuronal β-amyloid in neuronal cells (such as a reduction of at least 20%, at least 50%, at least 75%, at least 90%, at least 95%, at least 99%, or 100%, for example as compared to such intraneuronal β-amyloid in neuronal cells prior to administration of the compound(s) or as compared to no administration of the compound(s)), or regulation of mitochondrial dynamics.

In some aspects, the method includes treating a subject using one or more of the disclosed compounds, where treating the subject results in one or more of suppression of mitochondrial oxidative stress in oxytosis/ferroptosis (such as a reduction of at least 20%, at least 50%, at least 75%, at least 90%, at least 95%, at least 99%, or 100%, for example as compared to such mitochondrial oxidative stress in oxytosis/ferroptosis prior to administration of the compound(s) or as compared to no administration of the compound(s)), maintenance of mitochondrial calcium homeostasis in oxytosis/ferroptosis, modulation of oxidative phosphorylation system, restoration of mitochondrial bioenergetics (such as an increase of at least 20%, at least 50%, at least 75%, at least 90%, at least 100%, at least 200%, or at least 500%, for example as compared to such mitochondrial bioenergetics prior to administration of the compound(s) or as compared to no administration of the compound(s)), promotion of mitochondrial biogenesis (such as an increase of at least 20%, at least 50%, at least 75%, at least 90%, at least 100%, at least 200%, or at least 500%, for example as compared to such mitochondrial bioenergetics prior to administration of the compound(s) or as compared to no administration of the compound(s)), reduction of intraneuronal β-amyloid in neuronal cells (such as a reduction of at least 20%, at least 50%, at least 75%, at least 90%, at least 95%, at least 99%, or 100%, for example as compared to such intraneuronal β-amyloid in neuronal cells prior to administration of the compound(s) or as compared to no administration of the compound(s)), or regulation of mitochondrial dynamics.

In some aspects, the method comprises inhibiting oxytosis/ferroptosis in a subject, where inhibiting oxytosis/ferroptosis in the subject results in one or more of suppression of mitochondrial oxidative stress in oxytosis/ferroptosis (such as a reduction of at least 20%, at least 50%, at least 75%, at least 90%, at least 95%, at least 99%, or 100%, for example as compared to such mitochondrial oxidative stress in oxytosis/ferroptosis prior to administration of the compound(s) or as compared to no administration of the compound(s)), maintenance of mitochondrial calcium homeostasis in oxytosis/ferroptosis, modulation of oxidative phosphorylation system, restoration of mitochondrial bioenergetics (such as an increase of at least 20%, at least 50%, at least 75%, at least 90%, at least 100%, at least 200%, or at least 500%, for example as compared to such mitochondrial bioenergetics prior to administration of the compound(s) or as compared to no administration of the compound(s)), promotion of mitochondrial biogenesis (such as an increase of at least 20%, at least 50%, at least 75%, at least 90%, at least 100%, at least 200%, or at least 500%, for example as compared to such mitochondrial bioenergetics prior to administration of the compound(s) or as compared to no administration of the compound(s)), reduction of intraneuronal β-amyloid in neuronal cells (such as a reduction of at least 20%, at least 50%, at least 75%, at least 90%, at least 95%, at least 99%, or 100%, for example as compared to such intraneuronal β-amyloid in neuronal cells prior to administration of the compound(s) or as compared to no administration of the compound(s)), or regulation of mitochondrial dynamics.

Any method known to those of ordinary skill in the art can be used to assess the results of the aspects of the method of the present disclosure. In some aspects, the results are determined using assays performed using a hippocampal nerve cell line lacking CB1 and CB2 receptors.

In other aspects, the disclosed compounds are useful for treating or preventing a metabolic disorder, such as cardiovascular disease, or diabetes including type I and type II diabetes. In some aspects, the disclosed compounds can help reduce and/or ameliorate mitochondrial dysfunction related to such disorders. In some aspects, the methods includes reducing blood glucose in a subject using one or more of the disclosed compounds, such as a reduction of at least 20%, at least 50%, at least 75%, at least 90%, at least 95%, at least 99%, or 100%, for example as compared to blood glucose levels prior to administration of the compound(s) or as compared to no administration of the compound(s).

In other aspects, the disclosed compounds are useful for treating or preventing cancer and/or tumors, including solid tumors, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is a melanoma.

The disclosed compounds can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

In some aspects, the methods includes treating cancer and/or a metastasis in a subject using one or more of the disclosed compounds, such as a reduction in the size or volume of a tumor, and/or the number, size, and/or volume of one or more metastases, such as a reduction, of at least 20%, at least 50%, at least 75%, at least 90%, at least 95%, at least 99%, or 100%, for example as compared to the size or volume of a tumor, and/or the number, size, and/or volume of one or more metastases, prior to administration of the compound(s) or as compared to no administration of the compound(s).

### B. Dosages and Compositions

Also disclosed here are pharmaceutical compositions containing a compound disclosed herein. In some aspects, the composition comprises a pharmaceutically acceptable carrier and one or more compound disclosed herein. The composition may include one or more of the disclosed compounds in an amount of from 5 mg to 5000 mg or more. For example, the formulation may include one or more of the disclosed compounds in an amount of from 5 mg to 5000 mg, such as from 50 mg to 2000 mg, from 100 mg to 1000 mg, from 100 mg to about 2000 mg, from 100 mg to about 500 mg, from 250 mg to 2500 mg, from 500 mg to 1800 mg, or from 800 mg to 1700 mg.

In some aspects, the composition is provided such that the subject is administered one or more of the disclosed compounds in an amount of from 5 mg to 5000 mg per day, such as 5 mg, 25 mg, 50 mg, 100 mg, 200 mg, 300 mg, 400 mg, 450 mg, 500 mg, 550 mg, 600 mg, 700 mg, 750 mg, 800 mg, 850 mg, 900 mg, 1000 mg, 1100 mg, 1200 mg, 1300 mg, 1500 mg, 1600 mg, 1650 mg, 1700 mg, 1800 mg, 2000 mg, 2500 mg, 3000 mg, 3500 mg, 4000 mg, 4500 mg, or 5000 mg per day of the compound(s).

In some aspects, one or more of the disclosed compounds are administered to a subject in an amount of from 1 mg/kg/day to 50 mg/kg/day. For example, one or more of the disclosed compounds can be administered in an amount of from 5 mg/kg/day to 30 mg/kg/day, or 10 mg/kg/day to 20 mg/kg/day. In some aspects, one or more of the compounds disclosed herein is administered in an amount of from 10 mg/kg/day to 20 mg/kg/day. In some aspects, one or more of the compounds disclosed herein is administered in an amount of 10 mg/kg/day. In some aspects, one or more of the compounds disclosed herein is administered in an amount of 20 mg/kg/day.

A disclosed composition may be administered to the subject one per day or more that once per day, such as from 1 to 4 or more times per day.

The pharmaceutical composition may be in any form suitable for administration to a subject by any route of administration known in the pharmaceutical art, including, for example, oral, nasal, injection, ocular, topical, rectal, vaginal, etc. Administration by injection may be a systemic administration and includes i.v. or i.p. modes of administration, such as subcutaneous, intravenous, intramuscular, intrathecal or intraperitoneal injection. Exemplary formulations include, but are not limited to, a solution, emulsion, suspension, powder, tablet, pill, capsule, gel, or cream.

### C. Combinations of Therapeutic Agents

The disclosed compounds may be administered to a subject alone or in combination with one another, and/or in combination with other established therapies. In some aspects, one or more of the disclosed compounds are used in combination with other therapies useful for the disease or condition being treated, such as a neurodegenerative disease or condition in a subject. The compounds, or compositions thereof, may be administered simultaneously or sequentially in any order, by the same administration route or by different administration routes. For sequential administration, the compound(s) and/or therapy(s) may be administered such that an effective time period of at least one compound and/or therapy overlaps with an effective time period of at least one other compound and/or therapy.

Exemplary therapeutic agent(s) useful for administering in combination with the disclosed compounds include, but are not limited to, anti-amyloid therapies, such as Aducanumab or Lecanemab; Cholinesterase inhibitors, such as Donepezil (Aricept), Galantamine (Razadyne), or Rivastigmine (Exelon); or memantine.

### VI. Examples

Chemicals and Reagents. All solvents and reagents were purchased from commercial sources and were used without further purification. Analytical scale quantities of CBN, CBD, THC standards, and other natural and synthetic cannabinoids were from Cayman Chemical (Ann Arbor, MI). Preparative scale quantities of CBN, CP1, CP2, CP3, and CP4 were synthesized in-house. Olivetol, 5-(1,1-dimethyl-heptyl) resorcinol, citral, but-2-enal, 3-methyl-2-butenal, n-butylamine, iodine, Na₂SO₃, ethyl acetate, petroleum ether, toluene, glutamate, RSL3, and erastin were from Sigma-Aldrich (Saint Louis, MO). Resorcinol, 5-methylresorcinol, 5-ethyl-1,3-benzenediol, 5-propylbenzene-1,3-diol, 5-butylresorcinol, 5-heptylresorcinol, 5-pentadecylresorcinol, p-cymene, p,α,α-trimethylbenzyl alcohol, 1-(4-methylphenyl)-1-methylethylamine, linalool, (+)-limonene, α-terpineol, and TLC plates were from Thermo Fisher Scientific (Waltham, MA). Dowex 50WX8 (Cat #AAAL13921) was from VWR (Radnor, PA). Seahorse Xfe96 FluxPak (Cat #102416), Seahorse XF Cell Mito Stress Test Kit (Cat #103015), and Seahorse XF Real-Time ATP Rate Assay Kit (Cat #103592) were from Agilent Technologies (Santa Clara, CA).

General Instrumental Analysis. Optical absorbance and fluorescence were measured on a SpectraMax M5 Multi-Mode microplate reader (Molecular Devices, San Jose, CA).

Microscopy. Brightfield, phase contrast, and fluorescence microscopic images were acquired on an IX51 inverted microscope (Olympus Corporation, Tokyo, Japan) with an INFINITY3 monochrome CCD camera (Teledyne Lumenera, Ontario, Canada). Super-resolution microscopic images were acquired on a Zeiss LSM 880 rear port laser scanning confocal and Airyscan FAST microscope (Carl-Zeiss, Oberkochen, Germany). Image processing and analysis were performed with microscope software packages ZEN Black and ImageJ/Fiji.

Mass Spectrometry (MS). High-resolution mass spectrometric data were obtained on a Thermo Q-Exactive Quadrupole-Orbitrap mass spectrometer in positive mode. Samples were diluted with a 1:1 mixture of methanol and water containing 0.1% formic acid and then introduced by direct electrospray infusion. Accurate masses of all analytes were obtained from the pseudo-molecule [M+H]⁺ and were within 5 ppm mass error. Full MS scans were recorded for the 150-750 m/z range. MS/MS fragmentation was achieved by higher-energy collisional dissociation (HCD) at normalized collision energy settings between 10 and 30%.

Nuclear magnetic resonance (NMR). ¹H, ¹³C and 2D NMR data were collected at 298 K on a 600 MHz Bruker Avance III spectrometer fitted with a 1.7 mm triple resonance cryoprobe with z-axis gradients using TopSpin 3.6.0. NMR spectra were referenced to the residual solvent signal (δ_{H} 7.26, δ_{C} 77.2 for chloroform-d) with chemical shifts reported in δ units (ppm). Resonance multiplicities are denoted s, d, t, q, m, and br for singlet, doublet, triplet, quartet, multiplet, and broad, respectively. 2D HSQC and HMBC NMR spectra were collected using modified versions of the Bruker pulse sequences hsqcedetgpsisp2.3 and hmbcctetgpl3ndsp, which incorporated an ASAP module to enable faster data acquisition. Spectral widths were 12 ppm for the ¹H dimensions, 160 ppm for the HSQC ¹³C dimension and 250 ppm for the HMBC ¹³C dimension. For the HSQC spectra 8 scans and 256 t₁ increments were used. For the HMBC spectra 32 scans and 512 t₁ increments were used. Spectra were analyzed using Mnova.

In Silico ADME Prediction. SwissADME tool (Swiss Institute of Bioinformatics, www.swissadme.ch/) was used to compute physicochemical descriptors, predict ADME parameters, pharmacokinetic properties, druglike nature and medicinal chemistry friendliness of small molecules to support drug discovery.

Statistical Analysis. Data are presented as the mean±SD. The half maximal effective concentration (EC₅₀) was determined from sigmoidal dose response curves with four-parameter regression. The data were analyzed by one-way ANOVA with Tukey's multiple comparison post hoc test or Student's t test where appropriate. P values less than 0.05 were considered statistically significant (*p<0.05, **p<0.01, ***p<0.001, and ****p<0.0001). Analyses were performed using Excel and GraphPad Prism.

### Example 1

### Total Synthesis of Cannabinol (CBN)

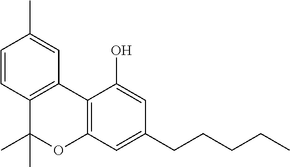 [SMILES: CCCCCc1cc(O)c2c(c1)OC(C)(C)c1ccc(C)cc1-2]

A total synthetic protocol was conducted according to modified literature methods disclosed by Liang et al. “Cannabinol inhibits oxytosis/ferroptosis by directly targeting mitochondria independently of cannabinoid receptors” Free Radic. Biol. Med. 2022, vol. 180, pp 33-51, and Caprioglio et al, “One-pot total synthesis of cannabinol via iodine-mediated deconstructive annulation” Org. Lett. 2019, vol. 21 (15), pp 6122-6125. Briefly, to a stirred solution of olivetol (1 g, 5.5 mmol), citral (910 μL, 5.5 mmol) and n-butylamine (530 μL, 5.5 mmol) were added in toluene (50 mL). The mixture was refluxed for 16 hours, and then cooled to room temperature. After Dowex 50WX8 (1 g) was added, the solution was stirred for 30 minutes at room temperature and then filtered. To the filtered solution, iodine (2.8 g, 11 mmol) was added, and the mixture was refluxed for an additional 5 hours. The reaction solution was quenched by adding saturated Na₂SO₃, extracted with ethyl acetate and then evaporated to dryness with a rotatory evaporator. The residue was reconstituted in 5% ethyl acetate/petroleum ether and then eluted on a HyperSep silica column (10 g) using the same solvents to give CBN as a yellow oil (1.3 g, 4.2 mmol, 76% total yield). The identity and purity of CBN were confirmed by high-resolution nuclear magnetic resonance (NMR) and mass spectrometry (MS) analyses.

Compound Characterization: ¹H NMR (600 MHz, CDCl₃) δ 8.18 (s, 1H), 7.15 (d, J=7.9 Hz, 1H), 7.08 (m, 1H), 6.45 (d, J=1.6 Hz, 1H), 6.29 (d, J=1.6 Hz, 1H), 5.29 (br s, 1H), 2.53-2.47 (m, 2H), 2.39 (s, 3H), 1.64-1.57 (m, 8H), 1.38-1.25 (m, 4H), 0.90 (t, J=6.9 Hz, 3H). ¹³C NMR (75 MHz, CDCl₃) δ 154.7, 153.1, 144.7, 137.0, 137.0, 127.7, 127.6, 126.5, 122.7, 110.9, 110.0, 108.8, 77.5, 35.7, 31.6, 30.6, 27.2, 27.2, 22.7, 21.7, 14.2. HR-MS m/z [M+H]⁺ 311.2009 (calculated for C₂₁H₂₇O₂⁺, 311.2006, 0.96 ppm error). Collected NMR and MS spectra for CBN were verified by cross-referencing with the reported data in the literature. The purity of CBN was over 95% as determined by quantitative NMR analysis.

### Example 2

### Total Synthesis of CP1 (2,2-dimethyl-7-(2-methyloctan-2-yl)-2H-chromen-5-ol)

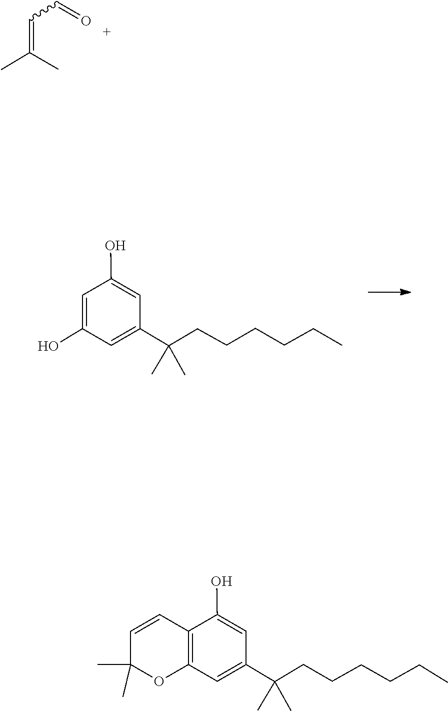

To a stirred solution of 5-(1,1-dimethyl-heptyl) resorcinol (0.50 g, 2.1 mmol), 3-methyl-2-butenal (231 μL, 2.75 mmol), and n-butylamine (201 μL, 2.75 mmol) were added in toluene (50 mL). The mixture was refluxed for 6 hours, and then cooled to room temperature. The reaction solution was evaporated to dryness with a rotatory evaporator. The residue was reconstituted in 5% ethyl acetate/petroleum ether and then eluted on a HyperSep silica column (10 g) using the same solvents to give CP1 as a yellow oil (0.54 g, 1.8 mmol, 85% total yield). The identity and purity of CP1 were confirmed by high-resolution NMR and mass spectrometry analyses.

Compound Characterization: ¹H NMR (600 MHz, CDCl₃) δ 6.63 (d, J=9.9 Hz, 1H), 6.43 (d, J=1.8 Hz, 1H), 6.32 (d, J=1.8 Hz, 1H), 5.56 (d, J=9.9 Hz, 1H), 1.54-1.49 (m, 2H), 1.46 (s, 6H), 1.26 (m, 2H), 1.21 (s, 6H), 1.21-1.19 (m, 4H), 1.08 (m, 2H), 0.87 (t, J=7.2 Hz, 3H). ¹³C NMR (75 MHz, CDCl₃) δ 153.3, 152.1, 150.1, 128.4, 116.4, 107.1, 106.9, 105.9, 76.0, 44.4, 37.7, 31.8, 30.0, 28.8, 28.8, 27.8, 27.8, 24.6, 22.6, 14.1. HR-MS m/z [M+H]⁺ 303.2313 (calculated for C₂₀H₃₁O₂⁺, 303.2324, −3.6 ppm error). The purity of CP1 was over 95% as determined by quantitative NMR analysis.

### Example 3

### Total Synthesis of CP2 (2-methyl-7-(2-methyloctan-2-yl)-2H-chromen-5-ol)

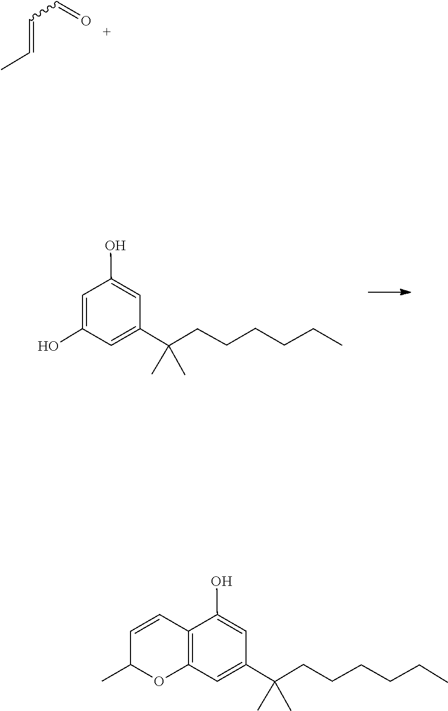

To a stirred solution of 5-(1,1-dimethyl-heptyl) resorcinol (0.50 g, 2.1 mmol), but-2-enal (193 μL, 2.75 mmol), and n-butylamine (201 μL, 2.75 mmol) were added in toluene (50 mL). The mixture was refluxed for 6 hours, and then cooled to room temperature. The reaction solution was evaporated to dryness with a rotatory evaporator. The residue was reconstituted in 5% ethyl acetate/petroleum ether and then eluted on a HyperSep silica column (10 g) using the same solvents to give CP2 as a yellow oil (0.52 g, 1.8 mmol, 87% total yield). The identity and purity of CP2 were confirmed by high-resolution NMR and mass spectrometry analyses.

Compound Characterization: ¹H NMR (600 MHz, CDCl₃) δ 6.70 (dd, J=9.8, 1.9 Hz, 1H), 6.44 (s, 1H), 6.34 (s, 1H), 5.59 (dd, J=9.8, 3.2 Hz, 1H), 4.97 (dq, J=3.2, 6.8 Hz, 1H), 1.55-1.50 (m, 2H), 1.48 (d, J=6.8 Hz, 3H), 1.26 (m, 2H), 1.21 (s, 6H), 1.21-1.19 (m, 4H), 1.08 (m, 2H), 0.87 (t, J=7.2 Hz, 3H). ¹³C NMR (75 MHz, CDCl₃) δ 154.1, 152.2, 151.1, 124.4, 118.4, 107.7, 106.5, 106.2, 71.3, 44.7, 37.8, 31.8, 30.1, 28.7, 28.7, 24.7, 22.7, 21.1, 14.1. HR-MS m/z [M+H]⁺ 289.2155 (calculated for C₁₉H₂₉O₂⁺, 289.2168, −4.3 ppm error). The purity of CP2 was over 95% as determined by quantitative NMR analysis.

### Example 4

### Total Synthesis of CP3 (2,2-dimethyl-7-pentyl-2H-chromen-5-ol)

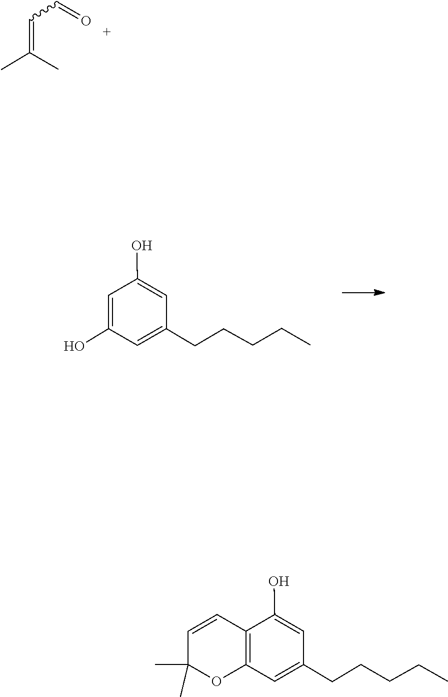

To a stirred solution of olivetol (0.5 g, 2.7 mmol), 3-methyl-2-butenal (231 μL, 2.75 mmol), and n-butylamine (201 μL, 2.75 mmol) were added in toluene (50 mL). The mixture was refluxed for 6 hours, and then cooled to room temperature. The reaction solution was evaporated to dryness with a rotatory evaporator. The residue was reconstituted in 5% ethyl acetate/petroleum ether and then eluted on a HyperSep silica column (10 g) using the same solvents to give CP3 as a yellow oil (0.62 g, 2.5 mmol, 91% total yield). The identity and purity of CP3 were confirmed by high-resolution NMR and mass spectrometry analyses.

Compound Characterization: ¹H NMR (600 MHz, CDCl₃) δ 6.64 (d, J=9.9 Hz, 1H), 6.28 (s, 1H), 6.20 (s, 1H), 5.53 (d, J=9.9 Hz, 1H), 2.44 (t, J=7.9 Hz, 2H), 1.57 (m, 2H), 1.43 (s, 6H), 1.33-1.24 (m, 4H), 0.91 (m, 3H). ¹³C NMR (75 MHz, CDCl₃) δ 153.5, 151.4, 144.7, 127.9, 116.7, 108.9, 108.2, 107.3, 76.0, 35.9, 31.5, 30.6, 27.7, 27.7, 22.5, 14.0. HR-MS m/z [M+H]⁺ 247.1687 (calculated for C₁₆H₂₃O₂⁺, 247.1698, −4.5 ppm error). The purity of CP3 was over 95% as determined by quantitative NMR analysis.

### Example 5

### Total Synthesis of CP4 (2-methyl-7-pentyl-2H-chromen-5-ol)

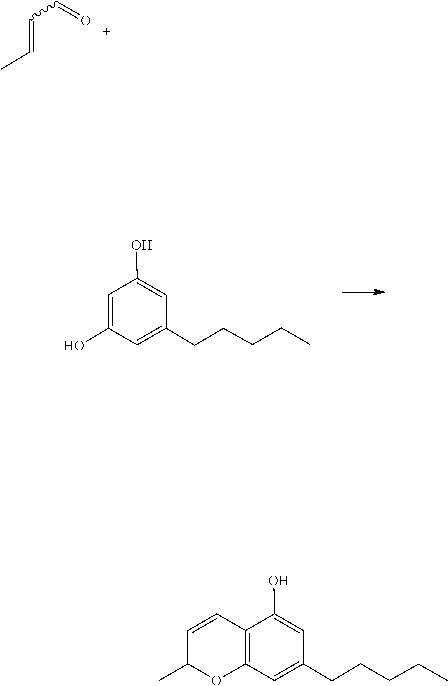

To a stirred solution of olivetol (0.5 g, 2.7 mmol), but-2-enal (193 μL, 2.75 mmol), and n-butylamine (201 μL, 2.75 mmol) were added in toluene (50 mL). The mixture was refluxed for 6 hours, and then cooled to room temperature. The reaction solution was evaporated to dryness with a rotatory evaporator. The residue was reconstituted in 5% ethyl acetate/petroleum ether and then eluted on a HyperSep silica column (10 g) using the same solvents to give CP4 as a yellow oil (0.58 g, 2.5 mmol, 92% total yield). The identity and purity of CP4 were confirmed by high-resolution NMR and mass spectrometry analyses.

Compound Characterization: ¹H NMR (600 MHz, CDCl₃) δ 6.79 (dd, J=10.0, 2.1 Hz, 1H), 6.38 (d, J=2.3 Hz, 1H), 6.26 (d, J=2.3 Hz, 1H), 5.61 (dd, J=10.0, 3.4 Hz, 1H), 4.98 (m, 1H), 2.47 (t, J=7.7 Hz, 2H), 1.59 (m, 2H), 1.49 (d, J=7.7 Hz, 3H), 1.41-1.28 (m, 4H), 0.95 (m, 3H). ¹³C NMR (75 MHz, CDCl₃) δ 153.8, 151.4, 144.8, 123.9, 118.4, 108.7, 108.6, 108.1, 71.3, 35.9, 31.4, 30.6, 22.5, 20.8, 14.0. HR-MS m/z [M+H]⁺ 233.1531 (calculated for C₁₅H₂₁O₂⁺, 233.1542, −4.5 ppm error). The purity of CP4 was over 95% as determined by quantitative NMR analysis.

### Example 6

### Fragment-Based Drug Discovery

A series of fragment chemicals with different variants of the substructures were assembled and screened in a cell-based phenotypic screening assays in the context of oxytosis/ferroptosis (FIG. 1). Specifically, for the left unit (LU), monoterpenoids with different functional groups and/or double-bond substitutions as well as ring arrangements were screened. For the central unit (CU), benzene rings with different hydroxyl group substitutions were screened. For the right unit (RU), different lengths, with or without branches, of the aliphatic hydrocarbon chains were examined.

After a number of fragment chemicals were screened, the SAR, basic chemical building blocks, and pharmacophores were assessed to determine which, if any, were active alone. This information was then used to identify the most active and viable substructure for each fragment. Combinations of substructures were used to form optimized CBN analogs through organic synthesis. The new CBN analogs were further evaluated for their physiochemical properties as well as their potency and efficacy in the in vitro and in vivo assays.

### I. Neuroprotective Effects of Fragment Compounds Against Oxytosis/Ferroptosis.

To determine the most active and viable substructure(s) responsible for their neuroprotective effects, the fragment compounds were first assayed for the inhibition of oxytosis/ferroptosis using two different cytotoxic insults (i.e., glutamate and RSL3). Compounds were tested at three different concentrations (1, 10, 50 μM) in order to cover weak to moderate bioactivities of the fragments. CBN, CBD and THC were used as reference compounds.

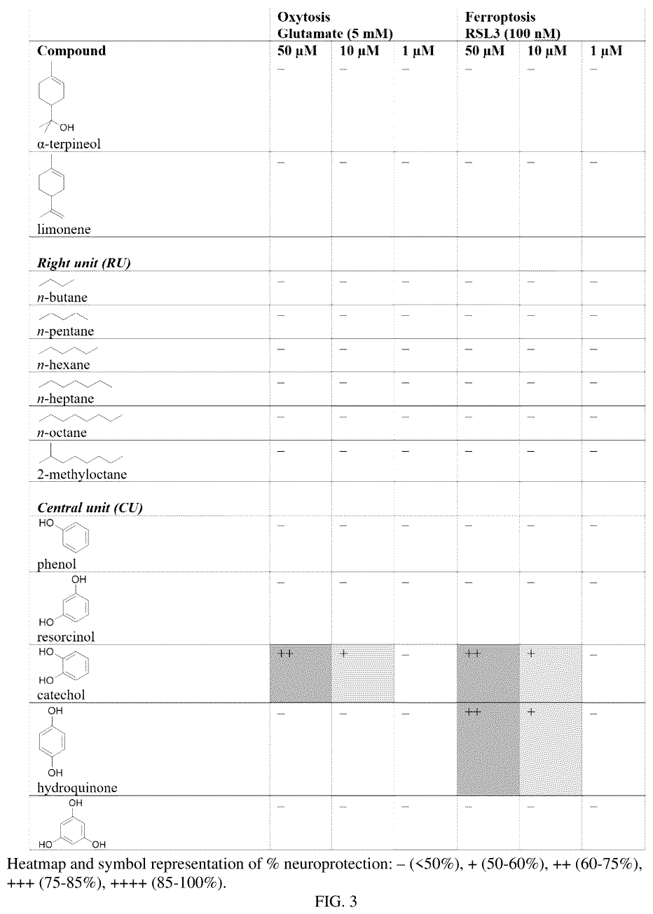

As summarized in FIGS. 2 and 3, for the left unit (LU) that represents cannabinoid-relevant monoterpenoids, none of them at concentrations ranging from 1-50 μM showed any neuroprotective activity in the assays. For the right unit (RU), different linear or branched aliphatic hydrocarbon chains (carbon numbers: C4-C9) were examined. As expected, these simple, water insoluble alkanes also showed no protective effects in the cell-based assays (FIG. 3). Together, these results suggested that, by themselves, neither the LU nor the RU fragment of CBN, CBD or THC was a viable pharmacophore.

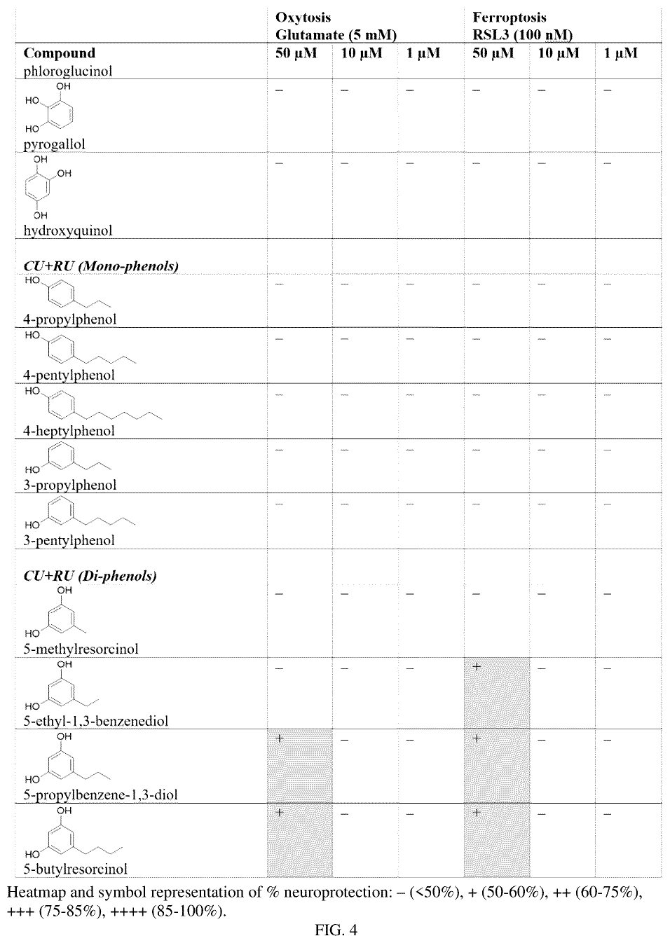

For the central unit (CU) in which the benzene ring is substituted with different numbers and positions of free hydroxyl (—OH) groups, it was found that in general these simple mono- (i.e., phenol), di- (i.e., resorcinol, catechol, or hydroquinone) and tri- (i.e., phloroglucinol, pyrogallol, or hydroxyquinol) hydroxy phenols at concentrations ranging from 1-50 μM showed limited neuroprotective activity against oxytosis/ferroptosis (FIGS. 3 and 4). The exceptions were the diphenols catechol (1,2-dihydroxybenzene) and hydroquinone (1,4-dihydroxybenzene) which both showed weak neuroprotective activities only at higher concentrations (10-50 μM). Nevertheless, the data suggested that the CU fragment containing functional antioxidant groups such as aromatic hydroxyls on the phytocannabinoids were a pharmacophore that may be useful for neuroprotection in the cell-based assays.

To further elaborate the role of the CU in neuroprotection, combinations of substructures were screen in cell-based assays (FIGS. 4 and 5). Both the LU and RU were biologically inactive in the assays. However, while the LU was structurally complex (e.g., containing cyclic structures and/or stereocenters), the RU was structurally simple (e.g., aliphatic hydrocarbon chains). As shown in FIGS. 4 and 5, the monophenols substituted with linear alkyl groups (C3, C5, and C7) showed no improvement in their neuroprotective activity. The diphenols substituted with short, linear alkyl groups (from C1 to C3) showed marginal improvements in their neuroprotective activity. However, the diphenols with longer, linear alkyl chains (from C4 to C7) incrementally increased their neuroprotective activity. For example, 5-pentyl-1,3-benzenediol (olivetol), 4-hexylbenzene-1,3-diol (4-hexylresorcinol), 4-butylbenzene-1,2-diol, and 2-tert-butylbenzene-1,4-diol showed good to excellent neuroprotective activity at 10-50 μM.

While resorcinol (benzene-1,3-diol) tended to gradually increase its neuroprotective activity as the length of its linear 5-alkyl chain was increased, the neuroprotection seemed to reach an optimum when the length of the 5-alkyl chain ranged from C5 to C7. Indeed, a much longer aliphatic hydrocarbon chain like 5-pentadecylresorcinol showed no further improvement in neuroprotective activity. Similar results were also seen with the bioisosteric variants of 5-alkylbenzene-1,3-diols such as 5-butoxybenzene-1,3-diol and 5-(pentyloxy)benzene-1,3-diol where the first carbon atom on the 5-alkyl chain was replaced by an oxygen atom. However, the inclusion of a branched 5-alkyl chain such as 5-(1,1-dimethylheptyl) resorcinol greatly improved the potency of the compound (>50% neuroprotection at 1 μM).

### II. Rational Design and in Silico Pharmacokinetic Evaluation on the New CBN Analogs.

Based on the resulting SAR data from the screening of the fragment compounds, new CBN analogs were designed by incorporating the CU with RU rather than LU on account of desirable bioactivity and synthetic accessibility. Two resorcinols were selected, olivetol and 5-(1,1-dimethylheptyl) resorcinol, as the candidate building blocks for the CU+RU. In addition, a portion of the LU of CBN was removed with the goal of decreasing the molecular weight and lipophilicity of the CBN analogs because these two molecular properties are the most important factors in influencing the pharmacokinetics and pharmacodynamics of drug candidates. Instead, a simplified version of the benzopyran ring system as the LU was designed in order to retain the structural rigidity and relevant bioactivity. Combinatorial chemistry was then performed (FIG. 15). A one-step total synthesis through amine-catalyzed reactions of resorcinols with α,β-unsaturated aldehydes (i.e., senecialdehyde and crotonaldehyde) resulted in the production of four new CBN analogs (CP1 to CP4) in high yields.

CBN and the new analogs were initially subjected to in silico pharmacokinetic evaluation using the SwissADME tool. As shown in Table 1, in general, the new analogs have a smaller molecular weight (MW) and lower lipophilicity (MlogP) relative to CBN. They also show an improvement in water solubility compared to CBN. Additionally, the four new analogs were predicted to be blood-brain barrier (BBB) permeable, which is advantageous for CNS drug development.

TABLE 1
Predicted physiochemical properties of CBN and the new analogs.

| | | | | HBDs | HBAs | Water | BBB |
|---|---|---|---|---|---|---|---|
| Compound | MW | MlogP | TPSA (Å²) | (n.OH, NH) | (n.O, N) | solubility | permeant |
| Desired CNS | ≤360 | ≤4.15 | ≤90 | ≤3 | ≤7 | soluble | yes |
| druglikeness | | | | | | | |
| CBN | 310 | 4.23 | 29.46 | 1 | 2 | poor | yes |
| CP1 | 302 | 4.17 | 29.46 | 1 | 2 | moderate | yes |
| CP2 | 288 | 3.94 | 29.46 | 1 | 2 | moderate | yes |
| CP3 | 246 | 3.23 | 29.46 | 1 | 2 | moderate | yes |
| CP4 | 232 | 2.98 | 29.46 | 1 | 2 | moderate | yes |

Desirable CNS drug-like properties include molecular weight (MW)≤360, calculated partition coefficient (MlogP)≤4.15, topological polar surface area (TPSA)≤90, number of hydrogen-bond donors (HBDs)≤3, and number of hydrogen-bond acceptors (HBAs)≤7 to improve penetration of the blood-brain barrier (BBB). Predictions were calculated with the SwissADME tool.

### Example 7

### Cell Culture

HT22 mouse hippocampal nerve cells were cultured in high-glucose Dulbecco's modified Eagle's medium (DMEM) (Invitrogen, Cat #11995065, Carlsbad, CA) supplemented with 10% fetal bovine serum (FBS) (Invitrogen, Carlsbad, CA) and 1% antibiotics including penicillin and streptomycin (Invitrogen Cat #10378016, Carlsbad, CA). Cell cultures were incubated at 37° C. in a fully humidified atmosphere containing 10% CO₂.

### Example 8

Neuroprotective effects of the new CBN analogs against oxytosis/ferroptosis.

### Oxytosis Assay

The assay procedure described in Fischer et al., “Old age-associated phenotypic screening for Alzheimer's disease drug candidates identifies sterubin as a potent neuroprotective compound from Yerba santa” Redox Biol. 21 (2019) 101089 was used. Briefly, HT22 cells were seeded at 3,000 cells/well in 96-well tissue culture plates in DMEM plus 10% FBS and 1% antibiotics. After 24 hours of plating, the cells were pretreated with different concentrations of the test compounds or a vehicle control for 1 hour followed by coincubation with 5 mM glutamate to initiate the cell death cascade. After 16 hours of treatment, cell viability was measured by the 3-(4,5-dimethylthiazol-2-yl)-2,5-diphenyltetrazolium bromide (MTT) assay. Optical absorbance was measured at 570 nm on a SpectraMax M5 microplate reader. Samples were analyzed in eight to sixteen wells per independent experiment (n=8-16). Results are presented as the percentage of the controls with vehicle alone. Results were verified by visual inspection of the cells under a microscope and are shown in FIGS. 2-6 and Table 2 below.

### Ferroptosis Assay

The assay procedure described in Fischer et al. was used. Briefly, HT22 cells were seeded at 3,000 cells/well in 96-well tissue culture plates in DMEM plus 10% FBS and 1% antibiotics. After 24 hours of plating, the cells were pretreated with different concentrations of test compounds or a vehicle control for 1 hour followed by coincubation with 50-100 nM RSL3 or 500 nM erastin to induce the cell death cascade. After 16 hours of treatment, cell viability was measured by the MTT assay. Optical absorbance was measured at 570 nm on a SpectraMax M5 microplate reader. Samples were analyzed in eight to sixteen wells per independent experiment (n=8-16). Results are presented as the percentage of the controls with vehicle alone. Results were verified by visual inspection of the cells under a microscope and are shown in FIGS. 2-6 and Table 2 below.

### Results

TABLE 2
SAR results for CP1, CP2, CP3, CP4 and CBN

| | RSL3 | Glutamate | Erastin | Aβ |
|---|---|---|---|---|
| | (50 nM) | (5 mM) | (500 nM) | toxicity |
| Compound | EC₅₀ (μM) | EC₅₀ (μM) | EC₅₀ (μM) | EC₅₀ (μM) |
| CP1 | 0.68 | 1.89 | 2.78 | 0.031 |
| CP2 | 0.92 | 2.89 | 3.36 | 0.097 |
| CP3 | 0.86 | 2.60 | 3.78 | 0.144 |
| CP4 | 0.83 | 6.63 | 8.08 | 1.60 |
| CBN | 0.69 | 1.89 | 2.33 | 0.014 |

The new analogs (CP1 to CP4) were screened along with CBN as a reference control in the oxytosis/ferroptosis assays to determine their neuroprotective activities against three distinct insults: RSL3, glutamate, and erastin. As shown in Table 2, the new analogs CP1-CP4 had strong neuroprotective effects against RSL3-induced oxytosis/ferroptosis with EC₅₀ values ranging from 0.68 to 0.92 μM. These potencies were comparable to that of CBN (EC₅₀, 0.69 μM). Similar neuroprotective effects of the new analogs were also seen against glutamate- or erastin-induced oxytosis/ferroptosis, although their EC₅₀ values of low micromolar levels were somewhat higher than those for protection against RSL3 toxicity. Among the four analogs, CP1 showed the strongest potencies against the three insults and its EC₅₀ values were comparable to those of CBN.

### Example 9

### Seahorse XF Analysis

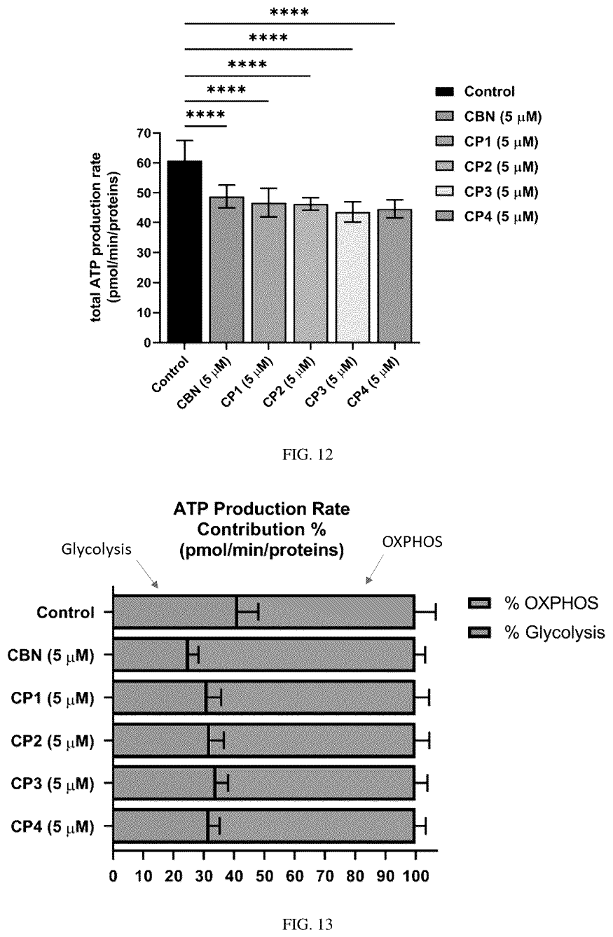

The assay procedure described in Liang et al., “Cannabinol inhibits oxytosis/ferroptosis by directly targeting mitochondria independently of cannabinoid receptors” Free Radic. Biol. Med. 180 (2022) 33-51 was used. Briefly, ATP production rates were assayed with a XF Real-Time ATP Rate Assay Kit using a Seahorse Xfe96 Extracellular Flux Analyzer (Seahorse Bioscience, North Billerica, MA). Complete Seahorse XF DMEM assay medium was supplemented with 10 mM glucose, 1 mM pyruvate and 2 mM L-glutamine, at pH 7.4. Mitochondrial ETC inhibitors were used at the following concentrations: 1.5 μM oligomycin and 0.5 μM of a 1:1 mixture of rotenone and antimycin A. Analyses were conducted using Wave software and XF Report Generators (Agilent Technologies). The sensor cartridge for the Xfe analyzer was hydrated overnight at 37° C. before the experiment. Data were normalized for total protein/well. Each condition was analyzed in 20-40 wells per independent experiment (n=20-40). FIGS. 12 and 13 provide the results.

For HT22 cells, 2,000 cells/well were seeded onto the Seahorse Xfe96 plates under normal culture condition as described above. The next day, cells were treated with test compounds at the desired concentrations and incubated for 16 hours. Immediately before the assay, the culture medium in the plates was replaced with complete Seahorse XF DMEM assay medium. The plates were incubated for 1 hour at 37° C. prior to the XF ATP rate tests according to the manufacturer's instructions.

### Results

CBN directly targets mitochondria and modulates their function including mitochondrial bioenergetics in cells and this effect of CBN is independent of canonical CB1/CB2 receptor signalling. CP1-CP4 along with CBN were assayed in the Seahorse XF real-time ATP rate and mitochondrial stress assays, which can be used to evaluate the phenotypic changes in mitochondrial metabolism upon compound treatment in cells.

As shown in FIG. 12, in HT22 neuronal cells the four analogs and CBN at 5 μM slightly but significantly decreased total ATP production rate to a similar level as compared to the control. In cells, ATP production is known to involve both mitochondrial oxidative phosphorylation (OXPHOS) and cytosolic glycolysis. CBN-treated cells showed an increase in the OXPHOS contribution to ATP production of approximately 15% relative to the control (FIG. 13), further indicating that CBN is a mitochondria-modulating compound. In comparison, the four analogs also showed similar modulatory effects on the upregulation of the mitochondrial OXPHOS contribution to ATP production by 10%.

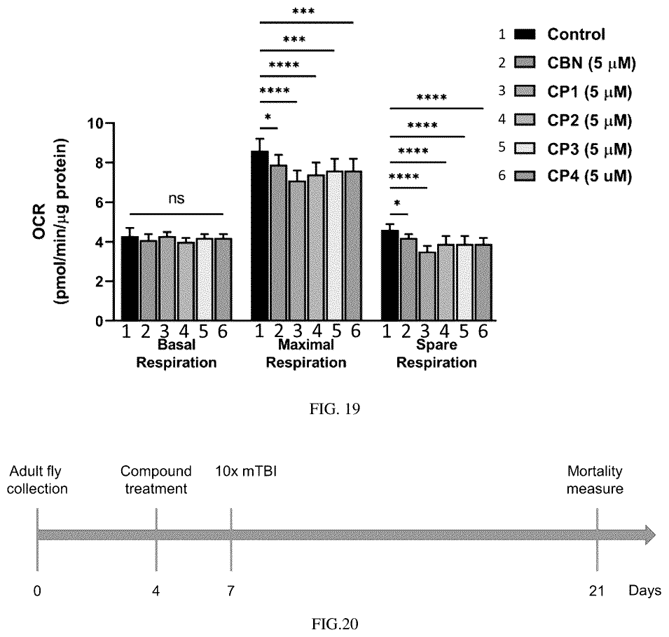

For the mitochondrial stress test (FIGS. 18 and 19), HT22 cells treated with CBN or the individual analogs showed a partial but significant decrease in maximal and spare mitochondrial respiration as compared with the control cells. Among them, CP1 showed the strongest effect on the suppression of maximal/spare OCR. In contrast, the basal respiration was not significantly affected by the compounds. Taken together, the Seahorse assay findings indicated that the CBN analogs had a similar mechanism of action as the parent compound CBN, specifically targeting mitochondrial function within cells.

### Example 10

Antioxidant effects of the new CBN analogs against oxytosis/ferroptosis.

### I. Mitochondrial Reactive Oxygen Species Measurement

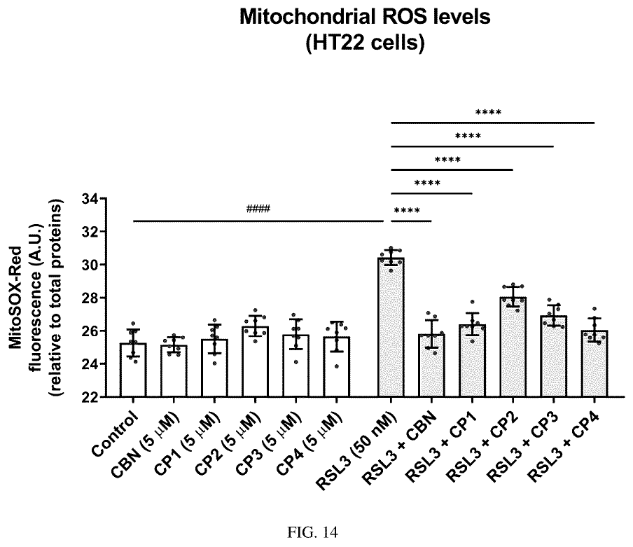

The assay procedure described in Liang et al. was used. Briefly, HT22 cells were seeded onto 96-well black walled plates at a density of 3,000 cells/well in DMEM supplemented with 10% FBS and 1% antibiotics. After the desired treatments, the mitochondrial superoxide ROS were detected with MitoSOX Red reagent (Ex/Em=510/580 nm). Experiments were performed according to the manufacturer's instructions. Fluorescence was measured on a SpectraMax M5 microplate reader. Data were normalized for total protein/well. Each condition was analyzed in eight wells per independent experiment (n=8). Results are presented as the percentage of the controls with vehicle alone. Results were verified by live-cell imaging under a fluorescence microscope. FIG. 14 provides the results.

### II. Lipid Peroxidation Measurements

The assay procedures were adapted from the procedures disclosed by Soriano-Castell et al., “Profiling the chemical nature of anti-oxytotic/ferroptotic compounds with phenotypic screening” Free Radic. Biol. Med. 177 (2021) 313-325 and Liang et al. “Cannabinol inhibits oxytosis/ferroptosis by directly targeting mitochondria independently of cannabinoid receptors” Free Radic. Biol. Med. 180 (2022) 33-51. Briefly, HT22 cells were seeded onto 96-well black walled plates at a density of 3,000 cells/well in DMEM supplemented with 10% FBS and 1% antibiotics. After the desired treatments for 16 hours, cells were labeled with 2.5 μM C₁₁-BODIPY 581/591 (oxidized form Ex/Em=488/520 nm) at 37° C. for 2 hours. Experiments were performed according to the manufacturer's instructions. Fluorescence was measured on a SpectraMax M5 microplate reader. Data were normalized for total protein/well. Each condition was analyzed in eight wells per independent experiment (n=8). Results were verified by live-cell imaging under a fluorescence microscope.

### III. Results

Activation of the oxytosis/ferroptosis pathway causes an elevation in cellular oxidative stress, particularly in mitochondria, where mitochondrial ROS (mtROS) contribute to the lipid peroxidation (LPO) of cellular membranes. The antioxidant parameters (i.e., anti-mtROS and anti-LPO) of the four new analogs were tested in comparison with CBN in HT22 cells treated with RSL3.

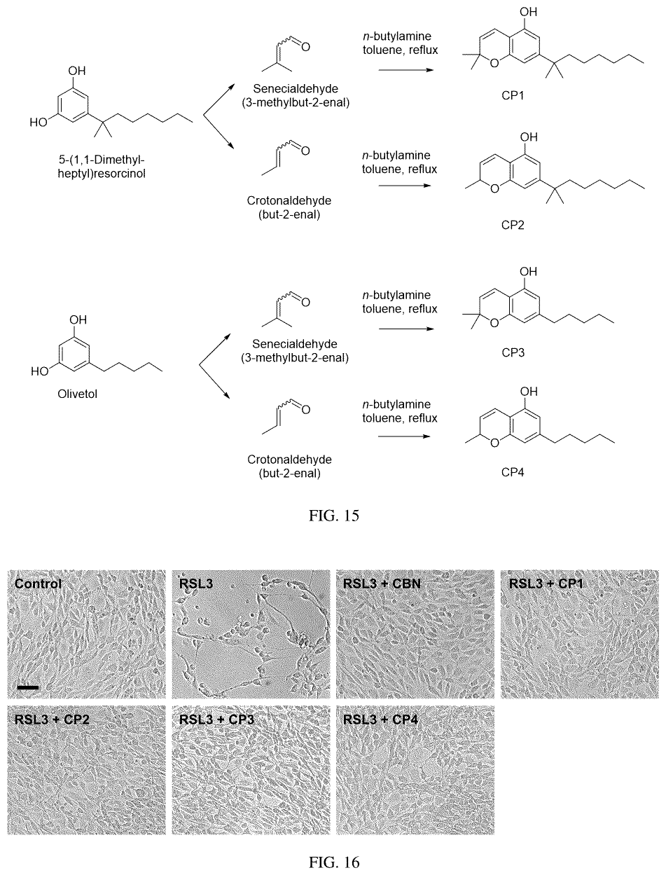

As shown in FIG. 16, microscopic imaging clearly showed that treatment with a subtoxic dose of RSL3 (50 nM) led to dramatic changes in cellular morphology with large numbers of rounded, shrinking, and detached cells present as compared to the control group. Cotreatment of RSL3 with 5 μM CBN or the individual analogs effectively prevented these morphological deteriorations in HT22 cells.

Further evaluation of the levels of oxidative stress (FIG. 14) indicated that the four analogs of CBN at 5 μM alone did not affect the redox status of mitochondria following treatment in HT22 cells. However, the cells treated with 50 nM RSL3 showed a significant increase in mtROS in comparison to the control cells (p<0.0001). Treatment with either CBN or the different analogs significantly suppressed RSL3-induced mtROS production. Among the four analogs, CP1 showed the strongest anti-mtROS effect, similar to that of CBN.

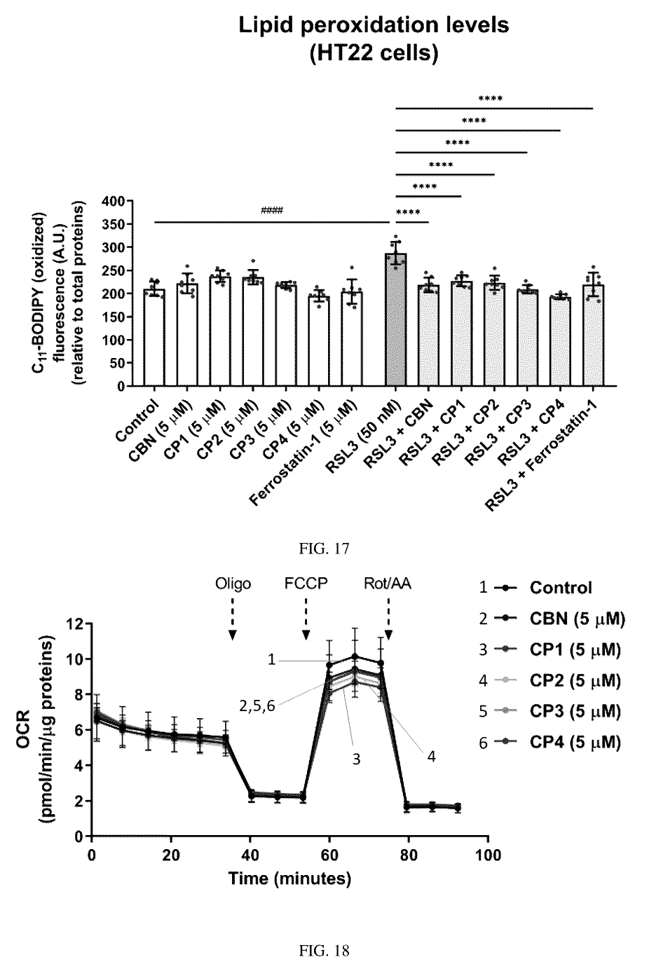

Regarding lipid peroxidation (LPO) (FIG. 17), the compounds (CBN and CP1-CP4) alone tested at 5 μM did not change the LPO levels in control HT22 cells. However, treatment with the analogs or CBN significantly suppressed the RSL3-induced increase in intracellular LPO (p<0.0001), which was similar to the effect of ferrostatin-1, a known LPO and oxytosis/ferroptosis inhibitor used as a reference control in the study. Together, the results demonstrated that the new analogs are very cytoprotective and effective at preventing increases in mtROS and LPO in cells induced by an activator of oxytosis/ferroptosis.

### Example 11

### Intracellular Amyloid Toxicity Assay

Accumulation of intracellular amyloid beta peptide (Aβ) is considered by many as being a primary toxic event in AD. The human nerve cell line MC65 conditionally expresses the C99 fragment of the amyloid precursor protein (APP) leading to the accumulation of intracellular AB. The MC65 cells are routinely grown in the presence of tetracycline and, following its removal, the expression of C99 is induced and the cells die within three days because of the accumulation of intracellular, toxic protein aggregates.

It was investigated whether the new analogs possessed similar protective profiles against intracellular amyloid toxicity in MC65 cells as compared to CBN. For the intracellular amyloid toxicity assay the procedure described in Huang et al., “Intracellular amyloid toxicity induces oxytosis/ferroptosis regulated cell death” Cell Death Dis. 11 (10) (2020) 828 was used. Briefly, MC65 cells were regularly grown in high glucose DMEM supplemented with 10% FCS and 2 μg/ml tetracycline. For the assay, cells were dissociated and plated at 1×10⁵ cells per well in 24 well tissue culture plates. After overnight cultivation in growth medium, the cells were washed three times with phosphate buffered saline containing calcium and magnesium and then switched to Opti-minimal essential media (Opti-MEM, Invitrogen) in the presence (no induction) or absence (APP-C99 induced) of 2 μg/ml tetracycline in the presence or absence different concentrations of the indicated compounds. At day 3, the control cells in the absence of tetracycline were dead, and cell viability was determined by the MTT assay. Optical absorbance was measured at 570 nm on a SpectraMax M5 microplate reader. Samples were analyzed in three wells per independent experiment (n=3). Results are presented as EC₅₀.

Results were verified by visual inspection of the cells under a microscope and are provided in Table 2. As shown in Table 2, the new analogs showed sub-micromolar potencies against intracellular amyloid toxicity (EC₅₀s from 0.031 to 1.6 μM) as compared with CBN (EC₅₀, 0.014 μM). Consistent with the data from the other screens, CP1 (EC₅₀, 0.031 μM) was the most potent analog in this assay.

### Example 12

Evaluation Using In Vivo Drosophila Mild Traumatic Brain Injury (mTBI) Model

### I. Drosophila Stocks and Culturing Conditions

The assay procedure described in Barekat et al., “Using Drosophila as an integrated model to study mild repetitive traumatic brain injury” Sci. Rep. 6 (1) (2016) 25252 and Candib et al., “The influence of cannabinoids on Drosophila behaviors, longevity, and traumatic injury responses of the adult nervous system” Cannabis and Cannabinoid Research (2023) were used. Briefly, adult wild-type Drosophila melanogaster Canton-S and w¹¹¹⁸ stock lines were obtained from the Bloomington Drosophila Stock Center. For all studies, Canton-S females were crossed with w¹¹¹⁸ males, to produce F1 outcrossed offspring (w¹¹¹⁸/+) along with established culturing conditions, including 25° C. and 60% humidity. Flies were collected 4 hours following eclosion using CO₂, aged as 25 flies per vial cohorts and maintained on standard fly media (molasses, cornmeal, agar, baker's yeast) for 1 week. Cohorts were transferred to vials containing standard fly media (0.1% ethanol), or defined dosages (μM) of compounds (0.1% ethanol). The dosage range for the compounds was based on published Drosophila findings. A 5 mg dose for adult flies is equivalent to about 16 μmol for the compounds or about 0.212 μM final concentration for a whole-body human (about 75 kg) dose. Flies were maintained on food containing CBN (3.0 μM) or the analogs (3.0 μM) for 3 weeks. To confirm palatability, flies were weighed 24 and 48 hours after being placed on the compound-containing food. No significant changes in weight were observed compared with non-treated controls. As a further control, flies were placed on compound-treated food containing blue dye #1. After 4 hours, 100% of flies had blue intestinal tracts.

II. Traumatic brain injury.

Traumatic brain injury (TBI) methods used for this study are described in Barekat et al. and Candib et al. Briefly, pretreated flies were anesthetized, placed in 2 mL screw cap tubes (10 flies/tube), and allowed to recover before being placed in the Omni Bead Ruptor-24 homogenizer (Omni International, Kennesaw, GA). Adult control and treated flies were treated for 3 days with different compounds before exposure to 10 mild trauma bouts (mild traumatic brain injury [mTBI] 10×, 2.1 m/s) at 1 week of age. Following injury, flies were returned to vials containing treated media and maintained using standard culturing conditions. The number of dead flies was counted starting 48 hours following trauma and then 3 times weekly for the remainder of the study. The percentage of dead flies described as the mortality index (MI) of treated cohorts after mTBI (10×) exposure for 3 weeks was calculated. Data were normalized for total flies/cohort. Each condition includes 110-235 flies per independent experiment (n=110-235).

### Results

Traumatic brain injury (TBI) is a complicated form of neurological disorder, and its pathological processes involve inflammation and mitochondrial dysfunction in brain cells. Emerging evidence suggests that TBI is also a risk factor for AD and other neurodegenerative diseases. Moreover, recent research reveals that the negative outcomes after TBI are potentially linked to activation of the oxytosis/ferroptosis cell death pathway. Thus, a Drosophila mTBI model was used to test the disclosed analogs in parallel with CBN and evaluate their in vivo neuroprotective efficacy (FIG. 20).

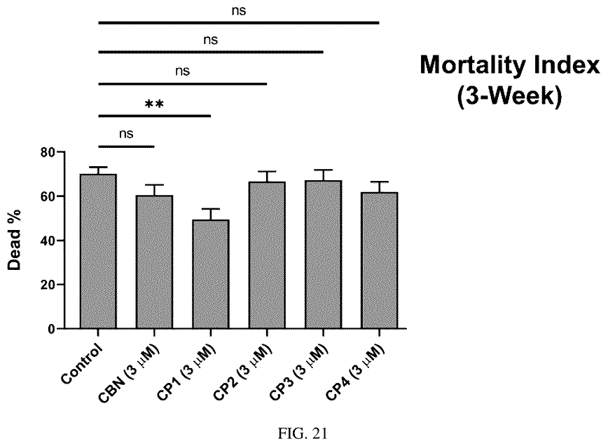

As shown in FIG. 21, approximately 70% of the vehicle-treated control flies died within 3 weeks after mTBI (10×) exposure. In contrast, there was a decrease in mortality (3-week mortality index [MI]) for all of the treatment cohorts (ranging from 49.5 to 67.3% MI). However, this decrease was only significant for the CP1-treated cohort where the MI went from 70.2% to 49.5% (p<0.0043). The protective effects of CBN (MI, 60.5%) and CP4 (MI, 61.9%) against mTBI-induced mortality were also notable although they did not reach statistical significance, while the effects of CP2 (MI, 66.7%) and CP3 (MI, 67.3%) were marginal.

### Discussion of Results from Examples 6-12

Oxytosis/ferroptosis is a novel form of regulated cell death that has gained increasing attention due to its possible involvement in many neurological disorders. In the inventors' continued efforts to identify natural product-based inhibitors of oxytosis/ferroptosis, a large number of natural and synthetic cannabinoids were screened using a cell-based phenotypic assays and it was found that the non-psychoactive phytocannabinoid, cannabinol (CBN), was highly neuroprotective. CBN showed sub-low micromolar potency (EC₅₀) against different inducers of oxytosis/ferroptosis and it appeared to do so by modulating multiple aspects of mitochondrial function. In addition, CBN had positive CNS druglike properties (MW=310, mLogP=4.23, PSA=29.46, HBD=1, HBA=2) along with good safety and pharmacokinetic profiles in humans. These findings suggested that CBN had promise as a drug lead for the treatment of neurological disorders. However, from a medicinal chemistry point of view, there remained multiple questions pertaining to the structural requirements and chemical space of CBN necessary for the inhibition of oxytosis/ferroptosis.

In the present study, a fragment-based method was used to systematically analyze the SAR of CBN. For this analysis, the structure of CBN was divided into three basic fragment units: a monoterpenoid fragment (left unit, LU), a phenol fragment (central unit, CU), and an aliphatic chain fragment (right unit, RU) (FIG. 1). A series of fragments with different variants of the basic substructures were first screened in a cell-based phenotypic assays for protection against oxytosis/ferroptosis. The results (FIGS. 2-5) clearly showed that neither the LU nor the RU alone was a bioactive pharmacophore of CBN. In contrast, the CU with functional antioxidant groups such as aromatic hydroxyls showed modest neuroprotection in a dose-dependent manner in the biological assays. Moreover, attaching a RU to the CU significantly increased the potency of this fragment against oxytosis/ferroptosis. Thus, the overall SAR analysis indicated that the CU was an important pharmacophoric element of CBN. Because the RU is a lipophilic, long-chain hydrocarbon, its addition to a CU may facilitate the improvement of compound potency by incrementally increasing cell membrane permeability and improving the physiochemical properties of the compound.

Based on the SAR information of CBN from the fragment-based drug discovery method, four new analogs, CP1 to CP4 (FIG. 15) were designed and synthesized. The structure of the newly designed analogs contains the CU+RU derived from either olivetol or 5-(1,1-dimethylheptyl) resorcinol, the two best 5-alkylbenzene-1,3-diols identified in the screening of fragment compounds (FIGS. 2-5). In addition, a simplified version of the benzopyran ring system was included as the LU in order to retain the structural rigidity and relevant bioactivity of CBN. In silico pharmacokinetic evaluation with the SwissADME tool suggested that these four analogs generally possessed better CNS druglike properties than CBN in terms of smaller molecular weights, a better balance of lipophilicity/hydrophilicity and BBB permeability (Table 1). The four CBN analogs also showed strong neuroprotection in the cell-based models against RSL3-, glutamate-, erastin- and Aβ-induced oxytosis/ferroptosis (Table 2). Among them, CP1 demonstrated EC₅₀ values ranging from 0.031 to 2.78 μM in the different neuroprotection assays, which were comparable to those of the parent compound CBN. These analogs, particularly CP1, also effectively prevented changes in cellular morphology and suppressed markers of cellular oxidative stress (i.e., mtROS and LPO) seen following induction of oxytosis/ferroptosis (FIGS. 14, 16 and 17).

Previously it was suggested that the neuroprotective effects of CBN against oxytosis/ferroptosis were dependent on the modulation of mitochondrial function. All four CBN analogs disclosed herein also appeared to directly target mitochondria in cells (FIGS. 12-13 and 18-19) where, similar to CBN, they slightly dampened mitochondrial oxygen consumption (i.e., maximal/spare OCR and total ATP production) but proportionally enhanced the mitochondrial OXPHOS capacity for ATP production. This shift in ATP generation between OXPHOS and glycolysis upon treatment with the compounds further supported a potential mode of action in terms of mitochondrial modulation and cellular bioenergetics. Taken together, the results suggested that these analogs partially reduced mitochondrial respiration and at the same time suppressed aberrant mitochondrial ROS production which consequently protected cells from ROS-mediated toxic lipid peroxidation and oxytosis/ferroptosis.

Traumatic brain injury (TBI) reflects an acute form of brain dysfunction caused by external forces to the head. In multiple models, TBI leads to elevated oxidative stress, lipid peroxidation, autophagic/lysosomal and mitochondrial dysfunction in brain cells as well as elevated levels of inflammation, with downstream effects including the loss of axonal connections and eventually nerve cell death. Epidemiological studies have consistently linked head trauma to a greater risk for AD and related dementias. Current evidence suggests a possible role for oxytosis/ferroptosis in TBI pathology and there is growing evidence that inhibition of oxytosis/ferroptosis can help to alleviate the long-term outcomes of TBI.

Drosophila melanogaster (fruit flies) have emerged as an excellent model organism for studying the mechanisms underpinning a range of neurodegenerative and neurological disorders. The Drosophila mTBI model can serve as a rapid, inexpensive, and highly effective tool to screen and evaluate the in vivo therapeutic potential of additional, cannabinoid-based compounds under conditions of acute and protracted neural stress. Here, CBN and its analogs were assayed using the Drosophila mTBI model to further assess their in vivo pharmacological efficacy. Over the 3-week study, none of the treatments exacerbated mortality rates but rather to varying degrees reduced the overall MI profiles of traumatized flies. One compound, CP1, showed significant beneficial effects for adult fly cohorts following mTBI exposure (FIG. 21). These in vivo results were consistent with the in vitro data that CP1 was the most efficacious of the four analogs in cell culture studies. Additionally, the trauma assay indicated that CP1 was more effective than CBN, while the two compounds had very similar profiles in cell-based assays. The improved activity of CP1 in fly trauma studies could reflect multiple whole animal features associated with pharmacokinetics such as drug absorption, distribution and metabolism that are not a part of the cell culture models of oxytosis/ferroptosis.

### Example 13

### Mouse Study

### Materials and Methods

Study Design: A group of 9-months-old female senescence-accelerated prone 8 (SAMP8) mice are fed with vehicle diet (LabDiet 5015, TestDiet, Richmond, IN), and treatment groups of 9-months-old female SAMP8 mice are fed with diets that include one of the disclosed cannabinol analog compounds at one of two dosage levels (LabDiet 5015+400 ppm cannabinol analog, LabDiet 5015+200 ppm cannabinol analog, TestDiet, respectively). Diet treatment lasts for four months until mice reached 13 months of age. At 9 months of age, SAMP8 mice already present a strong phenotype. Another group of 9-months-old female SAMP8 mice are used as the baseline control group. The effect of each of the disclosed cannabinol analog compounds is assessed in older SAMP8 mice after the four months of treatment and any age-related changes are defined by comparison to 9 months old SAMP8 mice. All mice are randomly assigned to experimental groups. The number of mice per group is determined based on sufficiency to attain statistical power. Behavioral testing is carried out one month prior to sacrifice and collection of biological material. Data is analyzed by blinded researchers when appropriate.

SAMP8 Mice. The SAMP8 line, a naturally occurring mouse line that was developed based on its phenotype of accelerated aging, is acquired from Harlan Laboratories (U.K.). Mouse body weights are measured regularly. All experiments are performed in accordance with the US Public Health Service Guide for Care and Use of Laboratory Animals.

Tissue Preparation: Mice are anesthetized and their blood collected by cardiac puncture. After perfusing with PBS, their brains are removed and dissected to collect cortex and hippocampus. Tissue samples are prepared for Western blotting, RNA extraction and metabolomic analysis.

Cell Lines: Mouse hippocampal HT22 cells are propagated as described by Davis J B, Maher P. 1994. Protein kinase C activation inhibits glutamate-induced cytotoxicity in a neuronal cell line. Brain Research 652:169-173. To prevent cell misidentification, large batches of each cell line are frozen that are regularly thawed to avoid using the wrong cell line. Cell lines are routinely tested for mycoplasma.

Primary Neurons: Primary cortical neurons are prepared from day 17 rat embryos and used at 7 days in vitro (7 DIV).

### Behavioral Assay

Elevated plus maze: The maze consists of four arms (two open without walls and two enclosed by 15.25 cm high walls) 30 cm long and 5 cm wide in the shape of a plus. A video-tracking system (Noldus EthoVision) is used to automatically collect behavioral data. The software is installed on a PC computer with a digital video camera mounted overhead on the ceiling, which automatically detects and records when mice enter the open or closed arms of the maze and the time spent in each. Mice are habituated to the room 24 hours before testing and are habituated to the maze for 1 minute before testing by placing them in the center of the maze and blocking entry to the arms. Mice are then tested for a 5-minute period and their behavior recorded. Disinhibition is measured by comparing the time spent on the open arms to time spent on the closed arms.

Barnes maze: The maze consists of a flat circular surface (36′ diameter) with 20 equally spaced holes (2′ diameter) along the outer edge. One of the holes leads to a dark hide box while the other 19 led to false boxes that are too small to be entered. The latency to enter the hide box is recorded. The test is conducted in three phases.

Phase 1 (Training): A hide box is placed under one of the holes. Animals are placed into an opaque cylinder in the center of the maze for 30 seconds to promote spatial disorientation at the start of the test. After 30 seconds, the cylinder is removed and the animal explores the maze until it finds and enters the hide box. The number of incorrect entries is scored. If the mouse fails to enter the box within 3 minutes, it is gently led into the box. The animal remains in the box for an additional 20 seconds before it is removed from the box and gently placed into the home cage. Training is repeated three times a day for four days. The location of the hide box remains the same during every trial but it is shifted between subjects to reduce the potential for unintended intra-maze cues.

Phase 2 (Retention): This phase measures retention of spatial memory following a delay. After a two-day break from training, each animal is re-tested for a one day, three-trial session using the same hide box location as before.

Phase 3 (Reversal): This phase examines memory reversal. On the day following the retention phase, a new hide box location is established 180 degrees to the original location. The same method as before is used and trials were repeated three times a day over two consecutive days.

### Study

Each of the disclosed cannabinol analogs is fed to aged SAMP8 mice and a multiomics approach is used to identify modes of action. The disclosed cannabinol analogs are investigated as to whether they reduce metabolic and gene transcription markers of aging in the SAMP8 model of aging and dementia when administered at a late stage of the aging process.

Testing Aging Associated with Changes in the Hippocampal Transcriptome

To identify age-dependent changes in brain metabolism that are causally associated with dementia, the disclosed cannabinol analogs are tested in SAMP8 mice. The SAMP8 mice are a model of accelerated aging that develop a progressive, age-associated decline in brain function as well as a number of brain pathologies similar to human dementia and AD patients. Treatment with the diets containing cannabinol start at 9 months of age and continued for four months (13 months of age); the median lifespan of these mice is 15 months. The entire transcriptome of brain hippocampal tissue is studied. Transcriptomic drift analysis, a method to characterize the aging process that measures changes in gene expression at a global level is done. The effects of the disclosed compounds on the expression of individual genes is examined.

Determination of improved cognitive function in SAMP8 mice when administered at advanced stages of dementia. Assessment of spontaneous behavior in the open field assay is accomplished between the 9 and 13 months SAMP8 mice. To investigate the effects of the disclosed cannabinol analogs on age-associated cognitive decline, mice are tested using the elevated plus maze and the Barnes maze, described herein. The elevated plus maze examines disinhibition behavior based on the aversion of normal mice to open spaces. Dementia is clinically associated with disinhibition and AD mouse models tend to exhibit increased disinhibition. The Barnes maze is used to analyze spatial learning and hippocampal-dependent memory. In this assay, mice use visual cues to locate a hidden box.

### VII. Exemplary Aspects

The following numbered paragraphs illustrate exemplary aspects of the disclosed technology.

Paragraph 1. A method, comprising administrating to a subject a compound having a structure according to Formula I

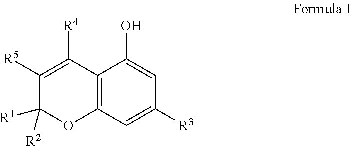

or a pharmaceutically acceptable solvate or prodrug thereof, wherein:
each of R¹ and R² independently is hydrogen or alkyl, where at least one of R¹ and R² is not hydrogen;
R³ is alkyl; and
each of R⁴ and R⁵ is hydrogen or alkyl.

Paragraph 2. The method of paragraph 1, wherein R³ is C₄₋₁₂alkyl.

Paragraph 3. The method of paragraph 1 or paragraph 2, wherein R³ is C₅-C₉alkyl.

Paragraph 4. The method of any one of paragraphs 1-3, wherein R⁴ and R⁵ are both hydrogen.

Paragraph 5. The method of any one of paragraphs 1-4, wherein each of R¹ and R² independently is hydrogen or C₁₋₆alkyl.

Paragraph 6. The method of paragraph 5, wherein each of R¹ and R² independently is hydrogen or methyl.

Paragraph 7. The method of paragraph 6, wherein both of R¹ and R² are methyl.

Paragraph 8. The method of any one of paragraphs 1-6, wherein R¹ is hydrogen and the compound has a structure according to Formula II

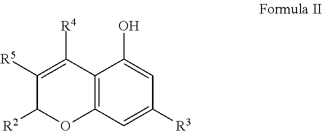

or a pharmaceutically acceptable solvate or prodrug thereof.

Paragraph 9. The method of paragraph 8, wherein the compound has structure according to Formulas iIa or iIb

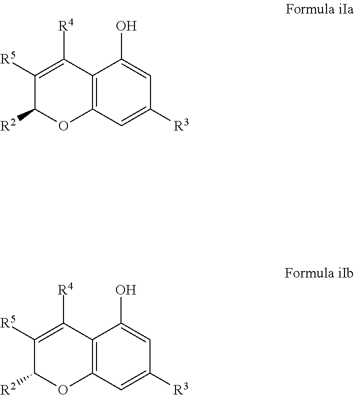

or a pharmaceutically acceptable solvate or prodrug thereof.

Paragraph 10. The method of paragraph 8 or paragraph 9, wherein R² is methyl.

Paragraph 11. The method of any one of paragraphs 1-10, wherein R³ is C₅-C₉ straight-chain alkyl, or C₅-C₉ branched alkyl.

Paragraph 12. The method of any one of paragraphs 1-11, wherein R³ is

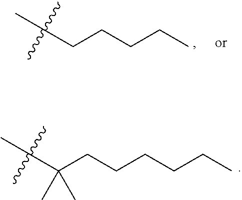

Paragraph 13. The method of paragraph 1, wherein the compound is selected from:
CP1: 2,2-dimethyl-7-(2-methyloctan-2-yl)-2H-chromen-5-ol;
CP2: 2-methyl-7-(2-methyloctan-2-yl)-2H-chromen-5-ol;
CP3: 2,2-dimethyl-7-pentyl-2H-chromen-5-ol;
CP4: 2-methyl-7-pentyl-2H-chromen-5-ol;
CP5: (S)-2-methyl-7-(2-methyloctan-2-yl)-2H-chromen-5-ol;
CP6: (R)-2-methyl-7-(2-methyloctan-2-yl)-2H-chromen-5-ol;
CP7: (S)-2-methyl-7-pentyl-2H-chromen-5-ol; or
CP8: (R)-2-methyl-7-pentyl-2H-chromen-5-ol.

Paragraph 14. The method of any one of paragraphs 1-13, wherein the compound is administered as a pharmaceutical composition comprising the compound and a pharmaceutically acceptable carrier.

Paragraph 15. The method of any one of paragraphs 1-14, wherein administering to the subject comprises treating or preventing a neurodegenerative disease or condition, a metabolic disorder, a traumatic brain injury, or cancer.

Paragraph 16. The method of paragraph 15, wherein the neurodegenerative disease or condition is Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), traumatic brain injury (TBI), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, or Icelandic hereditary cerebral hemorrhage with amyloidosis.

Paragraph 17. The method of paragraph 15, wherein the metabolic disease is diabetes or cardiovascular disease.

Paragraph 18. The method of paragraph 15, wherein the cancer is a cancer of the breast, prostate, colon, lung, skin, pancreas, liver, kidney, head and neck, or stomach.

Paragraph 19. The method of any one of paragraphs 1-18, wherein the subject is a mammal.

Paragraph 20. The method of any one of paragraphs 1-19, wherein the subject is a human.

Paragraph 21. The method of any one of paragraphs 1-20, wherein the method further comprises administering a second therapeutic agent.

Paragraph 22. A use of a compound having a structure according to Formula I, or a pharmaceutically acceptable solvate or prodrug thereof, in the manufacture of a medicament for treating a disease or condition

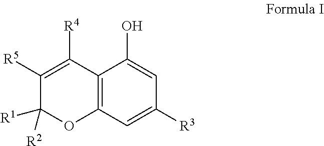

wherein:
each of R¹ and R² independently is hydrogen or alkyl, where at least one of R¹ and R² is not hydrogen;
R³ is alkyl; and
each of R⁴ and R⁵ is hydrogen or alkyl.

Paragraph 23. The use according to paragraph 22, wherein the disease or condition is a neurodegenerative disease or condition, a metabolic disorder, a traumatic brain injury, or cancer.

Paragraph 24. A compound having a structure according to Formula I, or a pharmaceutically acceptable solvate or prodrug thereof, for use in a method of treatment

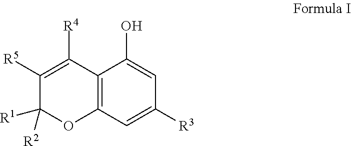

wherein:
each of R¹ and R² independently is hydrogen or alkyl, where at least one of R¹ and R² is not hydrogen;
R³ is alkyl; and
each of R⁴ and R⁵ is hydrogen or alkyl.

Paragraph 25. The compound for use according to paragraph 24, wherein the method of treatment is a method of treating or preventing a neurodegenerative disease or condition, a metabolic disorder, a traumatic brain injury, or cancer.

In view of the many possible aspects to which the principles of the disclosed technology may be applied, it should be recognized that the illustrated aspects are only examples of the disclosure and should not be taken as limiting the scope of the disclosure. Rather, the scope of the disclosed technology is defined by the following claims. We therefore claim as our disclosure all that comes within the scope and spirit of these claims.

We claim:

1. A method, comprising administrating to a subject a compound having a structure according to Formula I 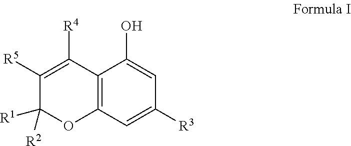
or a pharmaceutically acceptable solvate or prodrug thereof, wherein:
each of R¹ and R² independently is hydrogen or alkyl, where at least one of R¹ and R² is not hydrogen;
R³ is alkyl; and
each of R⁴ and R⁵ is hydrogen or alkyl.

2. The method of claim 1, wherein:
R³ is C₄₋₁₂alkyl;
R⁴ and R⁵ are both hydrogen; or
R³ is C₄₋₁₂alkyl and R⁴ and R⁵ are both hydrogen.

3. The method of claim 1, wherein each of R¹ and R² independently is hydrogen or C₁₋₆alkyl.

4. The method of claim 3, wherein each of R¹ and R² independently is hydrogen or methyl.

5. The method of claim 4, wherein R² is methyl.

6. The method of claim 4, wherein both of R¹ and R² are methyl.

7. The method of claim 1, wherein R³ is C₅-C₉ straight-chain alkyl, or C₅-C₉ branched alkyl.

8. The method of claim 1, wherein R³ is

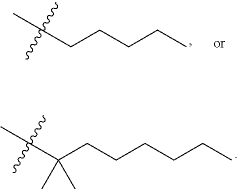

9. The method of claim 1, wherein R¹ is hydrogen and the compound has a structure according to Formula II 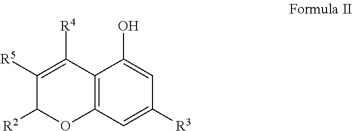
or a pharmaceutically acceptable solvate or prodrug thereof.

10. The method of claim 9, wherein the compound has structure according to Formulas IIa or IIb 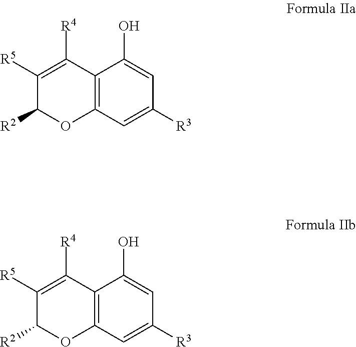
or a pharmaceutically acceptable solvate or prodrug thereof.

11. The method of claim 1, wherein the compound is selected from:
CP1: 2,2-dimethyl-7-(2-methyloctan-2-yl)-2H-chromen-5-ol;
CP2: 2-methyl-7-(2-methyloctan-2-yl)-2H-chromen-5-ol;
CP3: 2,2-dimethyl-7-pentyl-2H-chromen-5-ol;
CP4: 2-methyl-7-pentyl-2H-chromen-5-ol;
CP5: (S)-2-methyl-7-(2-methyloctan-2-yl)-2H-chromen-5-ol;
CP6: (R)-2-methyl-7-(2-methyloctan-2-yl)-2H-chromen-5-ol;
CP7: (S)-2-methyl-7-pentyl-2H-chromen-5-ol; or
CP8: (R)-2-methyl-7-pentyl-2H-chromen-5-ol.

12. The method of claim 1, wherein the compound is administered as a pharmaceutical composition comprising the compound and a pharmaceutically acceptable carrier.

13. The method of claim 12, wherein administering to the subject comprises treating or preventing a neurodegenerative disease or condition, a metabolic disorder, a traumatic brain injury, or cancer.

14. The method of claim 13, wherein the method reduces mortality by at least 20% as compared to no administration of the compound.

15. The method of claim 13, wherein the neurodegenerative disease or condition is Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), traumatic brain injury (TBI), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, or Icelandic hereditary cerebral hemorrhage with amyloidosis.

16. The method of claim 15, wherein the method reduces accumulation of intracellular amyloid beta peptide (AB), reduces mitochondrial ROS (mtROS), reduce lipid peroxidation (LPO), or combinations thereof, for example a reduction of at least 20% as compared to no administration of the compound.

17. The method of claim 13, wherein:
the metabolic disease is diabetes or cardiovascular disease; or
the cancer is a cancer of the breast, prostate, colon, lung, skin, pancreas, liver, kidney, head and neck, or stomach.

18. The method of claim 12, wherein the subject is a mammal.

19. The method of claim 12, wherein the subject is a human.

20. The method of claim 1, wherein the method further comprises:
administering a second therapeutic agent; or
performing an amyloid toxicity assay, a lipid peroxidation assay, and/or a mitochondrial ROS assay.
